(19) United States
(12) Patent Application Publication
(10) Pub. No.: US 2025/0332421 A1
(43) Pub. Date: Oct. 30, 2025

(54) SYSTEM AND METHOD FOR NERVE STIMULATION, CLOSED-LOOP FEEDBACK, AND PELVIC ORGAN PROLAPSE

(71) Applicant: Regenerative Bioelectronics Inc., Worcester, MA (US)

(72) Inventors: Mario Romero-Ortega, Coppell, TX (US); David Constantine, Sudbury, MA (US); Jayme Coates, Worcester, MA (US); Gregory Martin, Worcester, MA (US)

(21) Appl. No.: 19/180,108

(22) Filed: Apr. 15, 2025

Related U.S. Application Data
(60) Provisional application No. 63/634,422, filed on Apr. 15, 2024.

(51) Int. Cl. A61N 1/36; A61B 5/00; A61B 5/296; A61B 5/395; A61B 5/397; A61N 1/05; A61N 1/372

(52) U.S. Cl. CPC A61N 1/36139; A61B 5/296; A61B 5/395; A61B 5/397; A61B 5/4836; A61B 5/6847; A61B 5/7264; A61N 1/0558; A61N 1/36003; A61N 1/36007; A61N 1/3615; A61N 1/36171; A61N 1/36175; A61N 1/36178; A61N 1/37288

(57) ABSTRACT

A closed-loop neuromodulation system includes at least one implantable neurostimulator configured to deliver electrical stimulation to a target nerve innervating a muscle; at least one biosignal sensor configured to detect a physiological signal associated with the target muscle, wherein the physiological signal may include an electromyographic (EMG) or electroneurographic (ENG) signal; and a controller operatively coupled to the neurostimulator and the sensor. The controller is configured to receive the physiological signal from the sensor, determine whether the signal satisfies a predefined condition indicative of weakness, strength, fatigue, muscle underactivity, or overstimulation, and, in response to detecting that the condition is satisfied, automatically adjust one or more stimulation parameters selected from amplitude, frequency, pulse width, train duration, or train count.

### CROSS-REFERENCE TO RELATED APPLICATIONS

This patent application claims priority from provisional U.S. patent application No. 63/634,422, filed Apr. 15, 2024, entitled, “SYSTEM AND METHOD FOR NERVE STIMULATION, CLOSED-LOOP FEEDBACK, AND PELVIC ORGAN PROLAPSE,” and naming Mario Romero-Ortega, David Constantine, Jayme Coates, and Greg Martin as inventors, the disclosure of which is incorporated herein, in its entirety, by reference.

This patent application describes the use of an electrode stimulator device. In various embodiments, the electrode stimulator device may be any of the stimulator devices describes in U.S. patent application Ser. No. 16/185,285 [Attorney Docket No. 122289-20101], Ser. No. 16/414,169 [Attorney Docket No: 122289-20103], Ser. No. 18/225,129 [Attorney Docket No: 122289-10503], and/or Ser. No. 18/225,130 [Attorney Docket No: 122289-10603]. Each of these patent applications is incorporated herein by reference in its entirety.

The electrode stimulator device of various embodiments may be formed at least in part from a graphene fiber, which provides advantageous properties, as described in U.S. patent application Ser. No. 16/691,309 [Attorney Docket No: 122289-20301]. This patent application is incorporated herein by reference in its entirety.

The devices and methods described herein can be used to treat a number of conditions, including OAB, SUI, and/or FI, as described in in U.S. patent application Ser. No. 17/985,843 [Attorney Docket No: 122289-10403]. This patent application is incorporated herein by reference in its entirety.

### FIELD OF THE INVENTION

Illustrative embodiments of the invention generally relate to nerve stimulation and nerve recording and, more particularly, various embodiments of the invention relate to selective neuromodulation for controlling bladder and pelvic floor function.

### BACKGROUND OF THE INVENTION

Pelvic floor disorders, including urinary incontinence (UI), fecal incontinence (FI), and pelvic organ prolapse (POP), are prevalent conditions that significantly impact the quality of life, particularly for a substantial portion of the adult female population. These disorders often arise from injuries to pelvic nerves and muscles, which may occur during pregnancy, vaginal delivery, or as a result of surgical or obstetrical procedures involving the pelvic or perineal regions. Current treatment options for pelvic floor dysfunction are frequently limited in efficacy and may not provide adequate or lasting relief. Among these conditions, the simultaneous presence of both urinary and fecal incontinence—commonly referred to as double incontinence—represents one of the most severe and debilitating manifestations of pelvic floor dysfunctions.

### Summary of Various Embodiments

In accordance with an embodiment, a closed-loop neuromodulation system includes at least one implantable neurostimulator configured to deliver electrical stimulation to a target nerve innervating a muscle; at least one biosignal sensor configured to detect a physiological signal associated with the target muscle, wherein the physiological signal may include an electromyographic (EMG) or electroneurographic (ENG) signal; and a controller operatively coupled to the neurostimulator and the sensor. The controller is configured to receive the physiological signal from the sensor, determine whether the signal satisfies a predefined condition indicative of weakness, strength, fatigue, muscle underactivity, or overstimulation, and, in response to detecting that the condition is satisfied, automatically adjust one or more stimulation parameters selected from amplitude, frequency, pulse width, train duration, or train count.

In some embodiments, the sensor includes an intramuscular electrode configured to record EMG activity from one or more of the pubococcygeus, puborectalis, or iliococcygeus muscles. The predefined condition used by the controller may include a decrease in EMG envelope amplitude of 20% or more during stimulation, and upon detection of such a condition, the controller may reduce the stimulation amplitude to between 25% and 50% of a baseline threshold.

The system may further be configured to operate in a reflex stimulation mode, wherein stimulation is delivered to a sensory afferent to indirectly activate the target muscle. The controller may dynamically select between a direct stimulation mode and a reflexive stimulation mode based on factors such as the patient's condition, posture, bladder pressure, or reported symptoms.

In therapeutic use, the stimulation may be configured to treat pelvic organ prolapse (POP) by targeting nerves that innervate pelvic floor muscles such as the pubococcygeus, puborectalis, iliococcygeus, or coccygeus. Similarly, the stimulation may be configured to treat stress urinary incontinence (SUI) by targeting the pubococcygeus muscle via perineal nerve stimulation, or to treat overactive bladder (OAB) by adjusting stimulation to the perineal nerve or distal pudendal branches based on bladder pressure signals or detrusor EMG activity.

The stimulator may be configured to engage a distal portion of the target nerve comprising fewer than five fascicles and may be positioned to deliver stimulation to a segment located within the distal third of the anatomical length of the nerve.

In certain embodiments, the controller may be configured to switch from direct motor stimulation to reflexive sensory stimulation in response to a change in biosignal pattern, muscle condition, or patient posture. The system may also include intramuscular electrodes configured to record EMG activity specifically from the pubococcygeus, puborectalis, or iliococcygeus muscles, and may target perineal and/or pelvic floor muscles more broadly.

The controller may be further configured to titrate stimulation levels over time in accordance with progressive changes in the diagnosed physiological state of the muscle. This may involve logic that compares current biosignal features—such as peak amplitude or envelope—to baseline or prior-session values to determine whether the muscle is experiencing fatigue or strength gains.

Additionally, the controller may be programmed to maintain or reduce stimulation levels to avoid overstimulation or muscle fatigue. The system may be capable of initiating probe stimulations to determine motor response thresholds, using those results to adjust stimulation longitudinally as thresholds change over time.

In some embodiments, the controller determines trends in muscle function over multiple therapy sessions by analyzing historical biosignal data. Diagnosis may include evaluating shifts in motor response thresholds to assess whether a muscle has strengthened or become fatigued.

The system may be configured such that stimulation parameters are adjusted based on a physiological diagnosis rather than simply reacting to the detection of an individual event. To facilitate this, the system may periodically apply probe stimulations to assess motor response thresholds, using those assessments to guide long-term therapy titration.

In accordance with another embodiment, a neuromodulation system has a primary stimulator having a controller with communication circuitry, and at least one implantable satellite stimulator, each of which is configured to be coupled to a different peripheral nerve. The primary controller is configured to wirelessly transmit power and control signals to the satellite stimulators. Both the primary stimulator and the at least one satellite stimulator are configured to deliver electrical stimulation to their respective nerves, either in a coordinated manner or independently of one another.

In certain embodiments, each satellite stimulator includes a nerve clip electrode designed to engage a nerve with a fascicle count between one and five. The primary controller may also be configured to receive biosignal data from one or more satellite stimulators and, based on the received data, adjust stimulation parameters for the corresponding or a different satellite stimulator.

The satellite stimulators may be configured to deliver stimulation asynchronously to enable differential activation of various pelvic floor muscles. In some embodiments, one satellite stimulator comprises a sensing module capable of detecting EMG signals, while another satellite stimulator may be limited to stimulation functions only.

In various implementations, the primary controller is capable of executing therapy routines for pelvic organ prolapse by independently activating stimulators associated with the pubococcygeus and puborectalis muscles. The satellite stimulators may therefore be positioned and controlled to treat pelvic organ prolapse by selectively stimulating these muscles individually.

Additionally, the satellite stimulators may be configured to treat stress urinary incontinence by delivering bilateral stimulation to the perineal nerve or distal branches of the pudendal nerve. For treatment of overactive bladder, at least one satellite stimulator may receive feedback from a bladder pressure sensor, neural signals from the pelvic nerve, or EMG signals from the detrusor muscle, and dynamically adjust stimulation to the sacral or pudendal nerve based on that feedback.

The nerves targeted by the system may be selected from the group consisting of the pudendal nerve, perineal nerve, levator ani nerve, inferior rectal nerve, and coccygeal nerve. The stimulator may be configured to engage a distal portion of such a target nerve where the nerve comprises fewer than five fascicles and may deliver stimulation to a segment within the distal third of the anatomical length of the nerve.

The stimulation delivered by the system may operate with a frequency ranging from 20 Hz to 50 Hz, a pulse duration between 200 and 300 microseconds, and an amplitude in the range of 0.5 to 2.0 milliamps. The primary stimulator in the system may include an onboard power source.

In some embodiments, the primary stimulator and at least one of the satellite stimulators are configured to stimulate different pelvic floor muscles. At least one satellite device may include its own pulse generator and be batteryless, relying entirely on wireless power transfer from the primary stimulator.

The satellite devices may support bidirectional communication with the primary controller. Furthermore, the primary controller may modulate the timing or waveform shape of stimulation delivered across multiple satellite devices in accordance with a predefined therapy regimen.

The system is suitable for treating one or more clinical conditions selected from pelvic organ prolapse, overactive bladder, fecal incontinence, sexual dysfunction, or nerve injury. Additionally, the satellite devices are configured to be implanted at distal nerve branches and may vary in size depending on the anatomical characteristics of the target nerve structure.

In accordance with yet another embodiment, a neuromodulation system for treating pelvic organ prolapse (POP) in a subject includes a principal neuromodulation device that is configured for implantation and has a processor, a power source, and a plurality of wireless communication coils. The system further includes a plurality of implantable satellite neuromodulation devices, each of which is configured to interface with a distinct peripheral nerve and receive wireless power and control signals from the principal device. Additionally, the system includes at least one physiological sensor configured to detect a biosignal associated with pelvic floor function. The principal neuromodulation device is configured to receive the biosignal from the at least one sensor and, based on that biosignal, dynamically control the stimulation timing or parameters at one or more of the satellite neuromodulation devices.

In some embodiments, at least one of the satellite neuromodulation devices is configured to stimulate a distal pelvic nerve having a diameter between 100 microns and 1.5 mm. The principal neuromodulation device may be configured for implantation on a larger pelvic nerve, such as one having a diameter between 1 mm and 6 mm.

The wireless communication coils within the principal device may include three orthogonally arranged coils or a double-D coil configuration to facilitate robust and multidirectional communication. The biosignal detected by the system may be selected from the group consisting of electromyography (EMG), electroneurography (ENG), strain, movement, pressure, or electrochemical data.

In some versions of the system, at least one physiological sensor is configured as a clip or a needle probe that attaches to or embeds within a pelvic floor muscle to provide accurate biosignal input. The processor of the principal neuromodulation device is optionally configured to adjust one or more stimulation parameters delivered by a satellite neuromodulation device, including stimulation amplitude, pulse frequency, duty cycle, or pulse train count.

The system may be configured to deliver individualized stimulation to at least two different pelvic nerves, each corresponding to distinct pelvic floor muscles. Furthermore, the physiological sensor and at least one of the satellite neuromodulation devices may be independently implantable and capable of communicating wirelessly with the principal neuromodulation device.

The principal neuromodulation device may be further configured to coordinate therapy for one or more coexisting pelvic conditions selected from stress urinary incontinence (SUI), overactive bladder (OAB), or fecal incontinence (FI), using biosignal feedback from the same sensor or from different sensors within the system.

In accordance with another embodiment, a neuromodulation system for treating pelvic organ prolapse (POP) includes a first implantable stimulator configured to deliver electrical stimulation to a first nerve that innervates a first pelvic floor muscle selected from the group consisting of the pubococcygeus and puborectalis muscles. The system further includes a second implantable stimulator configured to deliver stimulation to a second nerve innervating a second pelvic floor muscle that is distinct from the first. A controller is operatively coupled to both stimulators and is configured to coordinate the delivery of stimulation based on a predefined therapy regimen tailored for POP.

In some embodiments, the controller alternates stimulation between the first and second stimulators to avoid synchronous overactivation and to promote balanced engagement of the corresponding pelvic floor muscles. Additionally, the controller may be configured to reduce the stimulation amplitude, pulse train count, or treatment frequency when it detects that the EMG signal amplitude has decreased by more than 20%, suggesting the onset of muscle fatigue or overstimulation.

The first and second nerves targeted by the stimulators may be selected from different pelvic region nerves, such as perineal branches of the pudendal nerve or branches of the levator ani nerve. The stimulators may engage distal portions of the respective nerves, specifically those with fewer than five fascicles, and stimulation may be delivered to a segment of the nerve located within the distal third of its anatomical length.

The therapy regimen delivered by the system may include sessions consisting of 2 to 4 stimulation trains per muscle, with a recovery interval of at least two minutes between each train. Stimulation may be delivered at a frequency ranging from 20 Hz to 50 Hz, a pulse duration between 200 and 300 microseconds, and an amplitude of 0.5 to 2.0 milliamps.

In some configurations, the system includes at least one biosignal sensor capable of detecting electromyographic (EMG) or electroneurographic (ENG) signals, which are used to dynamically adjust stimulation parameters based on detected muscle fatigue or activation thresholds. The first and second pelvic floor muscles targeted for stimulation are selected to generate a lifting or closing effect on pelvic organs, thereby supporting their anatomical positioning.

Additional biosignal sensors may be included to monitor EMG or ENG signals specifically from the pelvic floor muscles. These signals can be used by the controller to further adjust stimulation parameters, especially in response to indications of fatigue, insufficient activation, or abnormal strain.

The system may be configured to deliver stimulation from the two implantable stimulators either sequentially or in parallel, depending on the therapeutic needs, in order to restore or reinforce muscular support for the pelvic organs.

In accordance with another embodiment, A neuromodulation system includes a first nerve interfacing device and a second nerve interfacing device, each designed for implantation within a subject and positioned to interact with a nerve. Both devices have a stimulation electrode and a communication module capable of transmitting and/or receiving data signals. The communication module of the first device is configured to wirelessly communicate with the second device.

In some embodiments, the first device acts as a nerve stimulator while the second functions as a nerve sensor. Alternatively, at least one of the devices may be configured to both deliver stimulation and record signals from the same nerve. The stimulation electrode may be housed in a chamber specifically designed to isolate or focus the delivered electrical signal. The communication modules may include antennas to support wireless interaction, whether between two implanted devices or between an implanted device and an external controller.

At least one of the nerve interfacing devices may include a processor for managing stimulation signal parameters, as well as a memory device for storing protocols or sensed data. The processor may be used to adjust stimulation parameters such as amplitude, frequency, pulse duration, duty cycle, train count, or waveform shape. One or both devices may also include an energy storage component, such as a battery or capacitor.

The system may further include one or more physiological sensors for monitoring patient condition. These sensors may be implanted and may detect various biosignals, such as EMG, ENG, nerve conduction velocity, pressure, motion, relative position, electrochemical data, or even patient feedback. A processor may analyze the sensor signals to determine appropriate timing for stimulation and, in some cases, may derive stimulation parameters directly from the physiological signals. These parameters may be used for closed-loop control of a nerve, muscle, or organ.

A separate control device may be included in the system to coordinate stimulation therapy. This controller may be external to the patient or implanted within the body, potentially within a body cavity or the digestive tract.

In some implementations, the first device may include a wireless power transmitter, while the second device includes a power receiver. The second device may lack its own internal power source and rely entirely on power received wirelessly from the first device. At least one of the devices may include a processor that coordinates the timing of stimulation based on a signal received from the other. The two devices may be implanted at different anatomical locations to deliver therapy to distinct nerves. In certain embodiments, one device includes a biosignal sensor and transmits feedback to the other for use in closed-loop modulation.

In another embodiment, a neuromodulation system for treating pelvic organ prolapse (POP) includes a first nerve interfacing device with a stimulation electrode positioned to stimulate a first nerve innervating a pelvic floor muscle, such as the pubococcygeus or puborectalis. A second nerve interfacing device is configured to stimulate or sense activity from a second, distinct nerve. A processor coordinates the stimulation delivered from both devices in accordance with a predefined therapy regimen for POP, using input from at least one sensor that monitors a physiological condition and provides feedback for closed-loop modulation.

In some configurations, the sensor is capable of detecting EMG, ENG, or bladder pressure signals from the pelvic floor or nearby anatomy. Based on this feedback, the processor may adjust stimulation timing or amplitude in response to fatigue, signal degradation, or pressure changes. The system may also deliver asynchronous stimulation from the two devices to promote coordinated activation of multiple pelvic muscles. Stimulation may be specifically delivered to distal nerve segments comprising one to five fascicles, located in the final third of the nerve's anatomical length.

In accordance with another embodiment, a method of delivering closed-loop neuromodulation therapy to a subject includes delivering electrical stimulation to a pelvic nerve, recording one or more biosignals from a muscle innervated by that nerve, and modifying one or more stimulation parameters based on the recorded biosignals.

In some embodiments, modifying the stimulation parameters includes adjusting one or more parameters across multiple therapy sessions in response to longitudinal changes in the biosignals. These stimulation parameters may include amplitude, frequency, pulse duration, train duration, the number of stimulation trains per session, or the frequency of therapy sessions.

The recorded biosignals may include electromyographic (EMG) or electroneurographic (ENG) signals, and the adjustments to stimulation may be based on signal characteristics such as response latency, signal amplitude, or signal stability.

In further embodiments, the method includes independently modifying stimulation to a first pelvic nerve relative to a second pelvic nerve based on differences in muscle activation or recovery. For example, stimulation delivered to the second nerve may be increased while stimulation to the first nerve is reduced in response to detected signs of fatigue or overstimulation in the first nerve's corresponding muscle.

The stimulation pattern may also be progressively intensified if the recorded biosignals indicate improved muscle recruitment or activation. This may be demonstrated through improvements such as reduced response latency, increased amplitude of biosignals, or increased strain response.

In some implementations, the method includes adjusting the timing or intensity of therapy delivery to prevent or minimize muscle fatigue, overstimulation, or neuromuscular imbalance.

A method of treating a pelvic health disorder includes electrically coupling a neuromodulation device to a nerve target that directly or reflexively innervates a target muscle, and stimulating the nerve target using the neuromodulation device. In some embodiments, stimulating the nerve includes delivering a probe stimulation to the nerve target, detecting a physiological response evoked by that probe stimulation, and using the evoked response as closed-loop feedback to adjust stimulation parameters for future therapy.

The method may be used to treat pelvic organ prolapse (POP), with target nerves including one or more of the pubococcygeus nerve, puborectalis nerve, iliococcygeus nerve, coccygeus (ischiococcygeus) nerves, inferior rectal nerve, and/or perineal nerve branches, particularly distal ones. In certain embodiments, overactive bladder (OAB), stress urinary incontinence (SUI), fecal incontinence (FI), and/or POP may be treated simultaneously using multiple neuromodulation devices.

Coupling the neuromodulation device may involve positioning the device along the last quarter or last third of the length of the target nerve. In some embodiments, the device is coupled to a portion of the nerve having between one and three fascicles. The neuromodulation device may include a multi-input, single-output (MISO) proportional-integral-derivative (PID) controller.

The neuromodulation device may include a hermetically sealed housing containing electronics, surrounded by a buffer layer, and include an elastomeric arm that defines a nerve channel along a longitudinal axis. The system may include a nerve stimulation chamber defined in part by the arm, and the chamber may retain a nerve in contact with an internal electrode.

In some embodiments, the method includes reducing the maximum cross-sectional dimension of a nerve to create a stretched, narrowed nerve; advancing the stretched nerve through the channel where wall pressure remains below 6.7 kPa at any point; positioning the nerve in the stimulation chamber; allowing it to expand; and maintaining at least 20% of the nerve's perimeter in contact with the electrode.

Illustrative embodiments applicable to the field of nerve stimulation and nerve recording and are applicable to sensory, motor and autonomic nerves within humans and animals.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows a patient in accordance with illustrative embodiments.

FIGS. 1B-1D schematically show details of various embodiments of neuromodulation devices in accordance with illustrative embodiments.

FIG. 1E depicts an implantable wireless electromyogram (EMG) sensor in accordance with illustrative embodiments.

FIG. 1F depicts an implantable nerve conduction velocity sensor arrangement in accordance with illustrative embodiments.

FIG. 1G depicts an implantable pressure sensor in accordance with illustrative embodiments.

FIG. 1H depicts an implantable movement sensor in accordance with illustrative embodiments.

FIG. 1I depicts an implantable relative movement sensor in accordance with illustrative embodiments.

FIG. 1J depicts an implantable tissue strain sensor in accordance with illustrative embodiments.

FIG. 1K depicts an implantable wireless electrochemical sensor in accordance with illustrative embodiments.

FIG. 1L depicts an implantable relative position sensor in accordance with illustrative embodiments.

FIG. 1M schematically shows an anatomical drawings of nerves branching towards various pelvic floor muscles in accordance with illustrative embodiments.

FIG. 1N shows a chart of nerve targets for treatment of various conditions in accordance with illustrative embodiments.

FIG. 2A shows a process for closed-loop neuromodulation in accordance with illustrative embodiments.

FIG. 2B shows the innervation patterns of a target nerve in accordance with illustrative embodiments of the invention.

FIGS. 3A-3B schematically show a system for neuromodulation in accordance with illustrative embodiments.

FIG. 4 schematically shows details of the neuromodulation device controller in accordance with illustrative embodiments of the invention.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

In illustrative embodiments, one or more miniaturized neuromodulation devices individually control pelvic and perineal nerves to treat stress urinary incontinence (SUI), fecal incontinence (FI), and/or overactive bladder (OAB). Illustrative embodiments enhance the regeneration of nerves and/or the nerve connection to affected tissue (e.g., muscle) using the neuromodulation devices. After nerve function and nerve connection to tissue is restored, applicable tissue functionality can be restored (e.g., muscles are strengthened). In the case of OAB, illustrative embodiments efferently stimulate the nerve to block (i.e. introduce interference to obfuscate) the bladder's urgency signals to the brain.

Furthermore, pelvic floor muscles support pelvic organs. Their function is maintained by complex interactions among levator ani muscles, the vagina, and connective tissue of the pelvic floor. These interactions are integrated and highly coordinated. Pelvic organ prolapse (POP) occurs when one or more of the pelvic organs, such as the bladder, uterus, or rectum, descend from their normal position and protrude into the vagina, often due to weakened pelvic muscles or connective tissue. Achieving a coordinated closure of the rectal and urethral sphincters, a restorative therapy to repair the lost support in POP, as well as to provide therapy for multiple conditions including SUI, OAB and FI, requires highly precise and specific coordinated activation of individual pelvic floor muscles and nerves. Illustrative embodiments use a network of sensing and stimulating neuromodulation devices that can sense and detect functional activity thresholds and deliver a coordinated multi-organ integrated response.

A closed-loop system of neuromodulation devices and sensors enables dynamic, feedback-responsive neuromodulation for conditions such as stress urinary incontinence (SUI), pelvic organ prolapse (POP), and overactive bladder (OAB). These embodiments utilize biosignals such as electromyography (EMG) or electroneurograms (ENG) to modulate stimulation parameters in real-time, adapting pulse amplitude, frequency, and duration based on muscle response, fatigue, or nerve integrity. This approach improves therapeutic efficacy while mitigating risks associated with overstimulation, such as muscle fatigue or nerve injury.

Various embodiments provide a distributed or “satellite” neuromodulation architecture, in which multiple miniaturized implantable devices—each targeting distinct nerve sites—operate under the coordination of a central controller. This configuration allows for synchronized or independent stimulation of multiple anatomical regions, such as different pelvic floor muscles or bilateral nerve targets. The satellite system supports wireless communication, power sharing, and modular therapy logic, making it particularly well-suited for anatomically complex or multi-site disorders like POP. Together, these innovations offer a flexible, scalable platform for personalized neuromodulation therapy with broad clinical utility. Details of illustrative embodiments are discussed below.

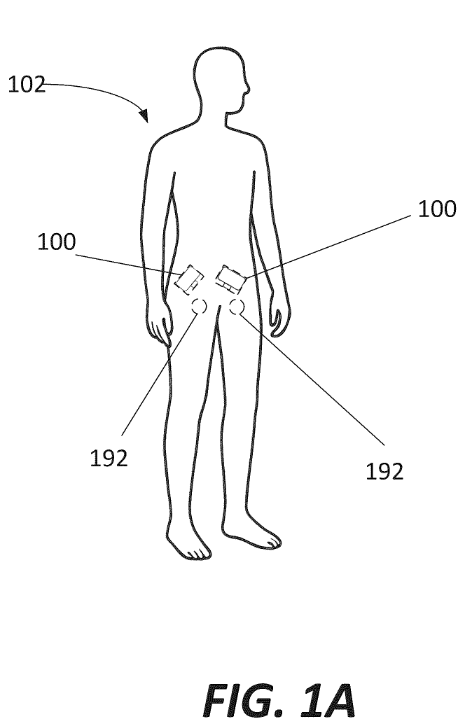

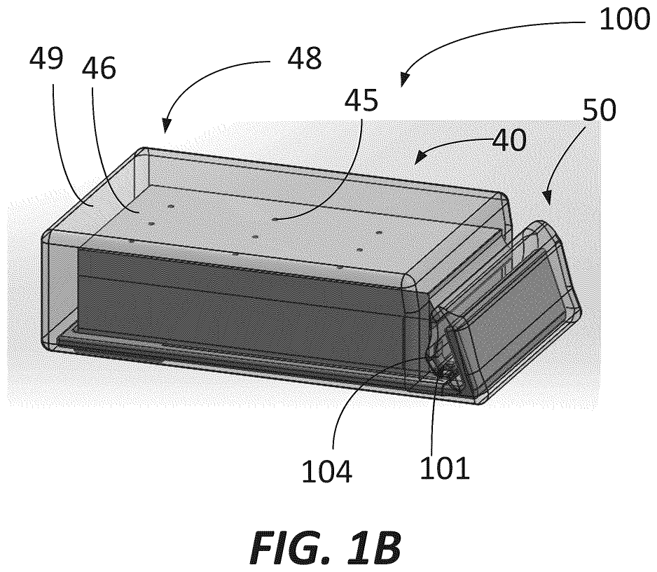

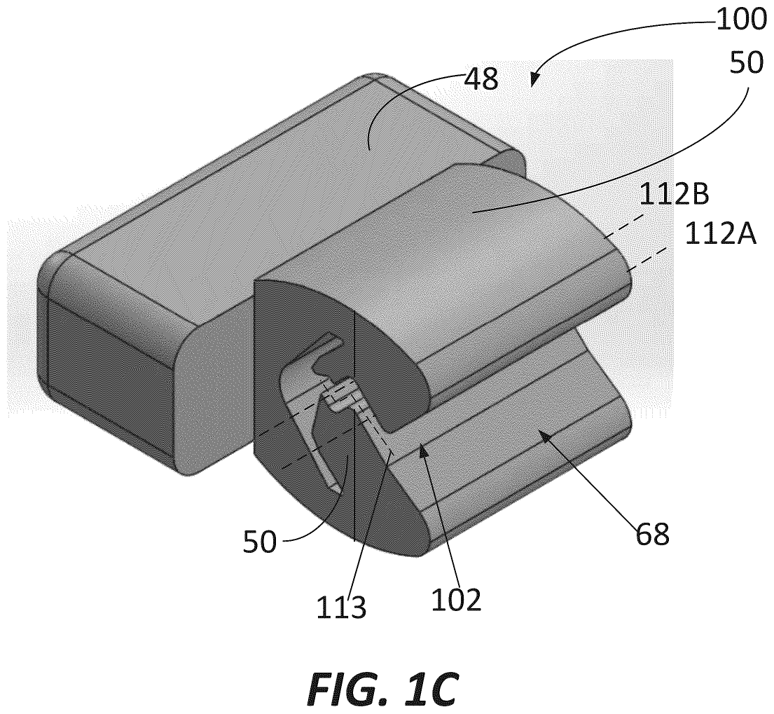

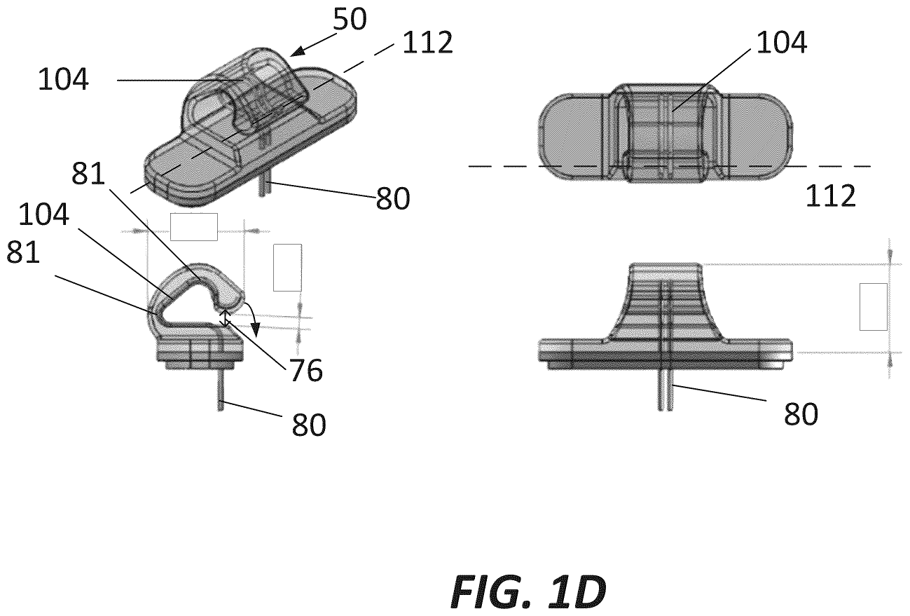

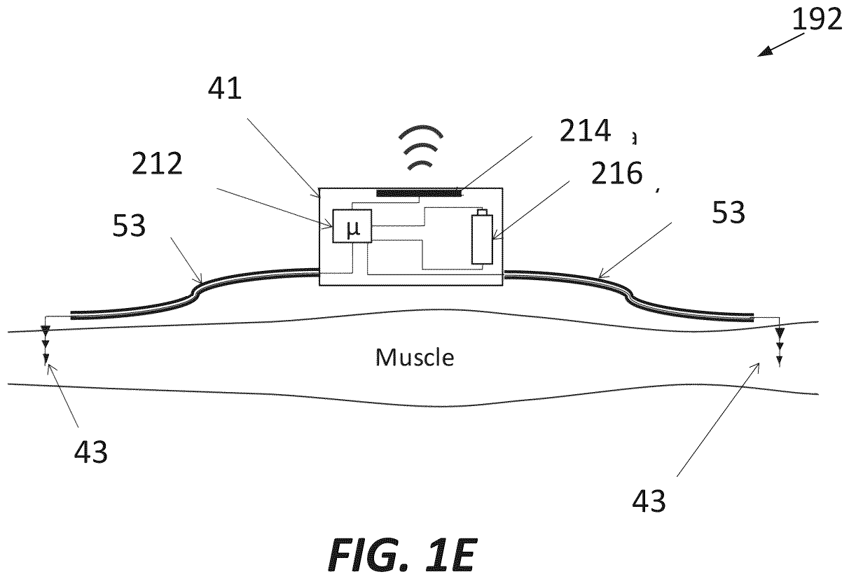

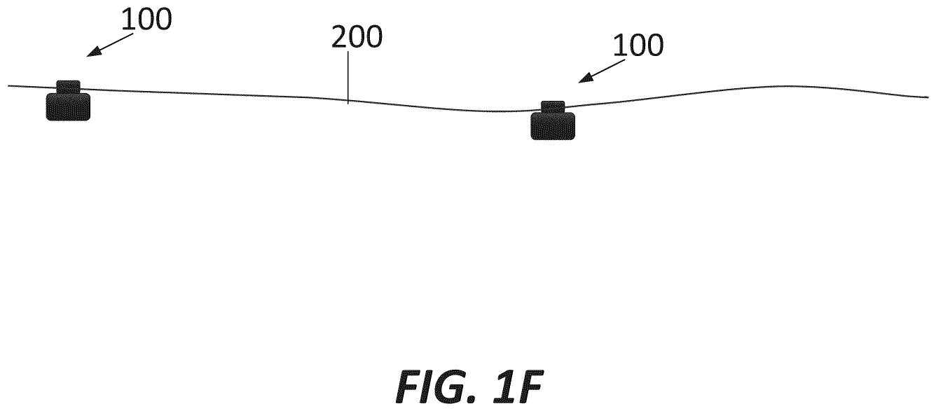

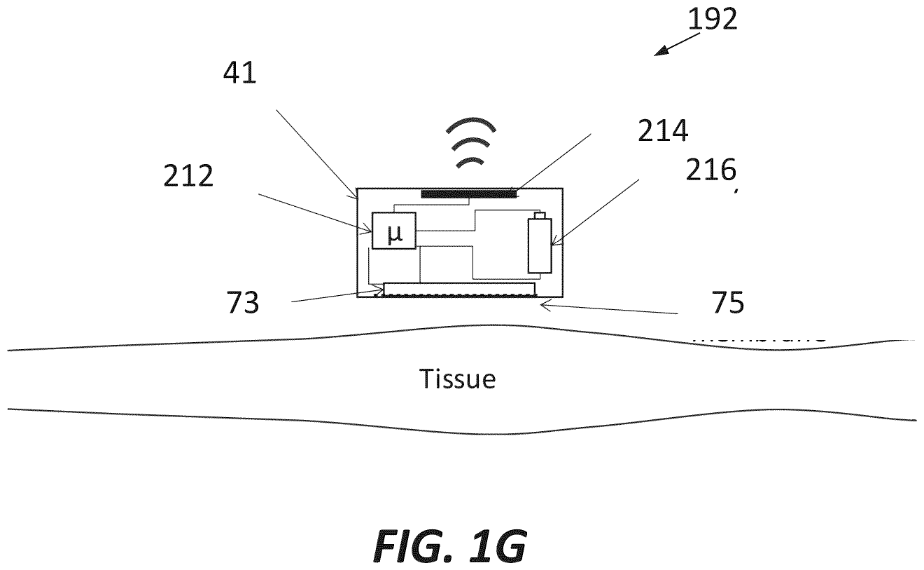

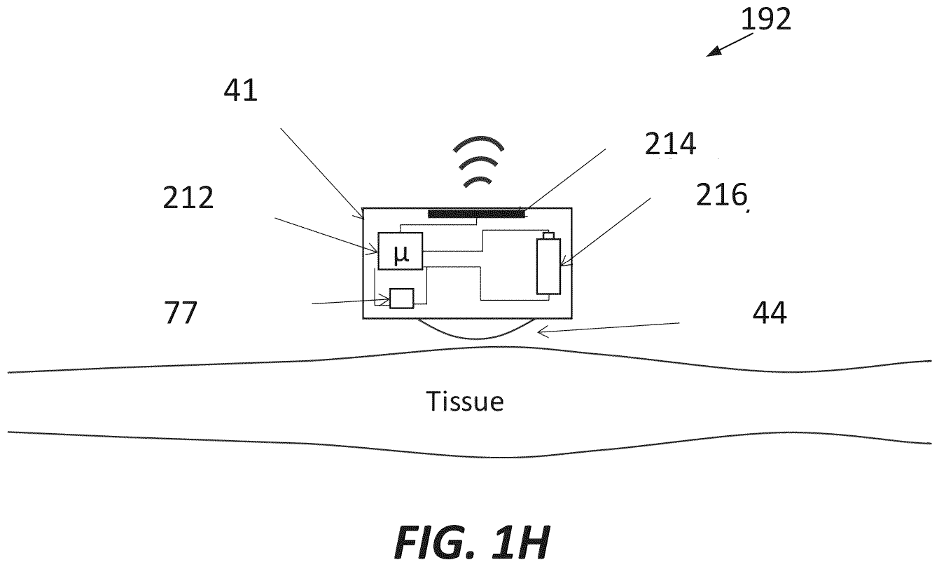

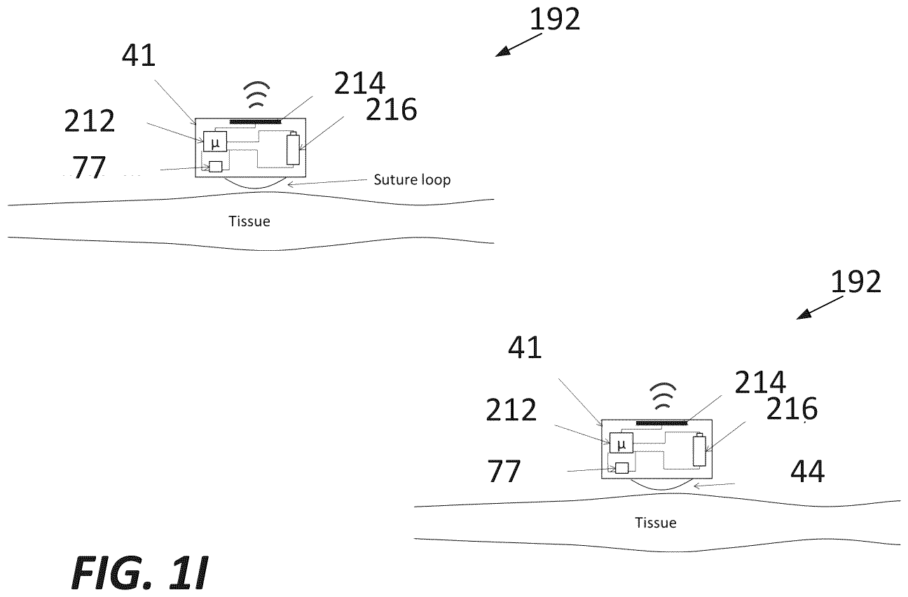

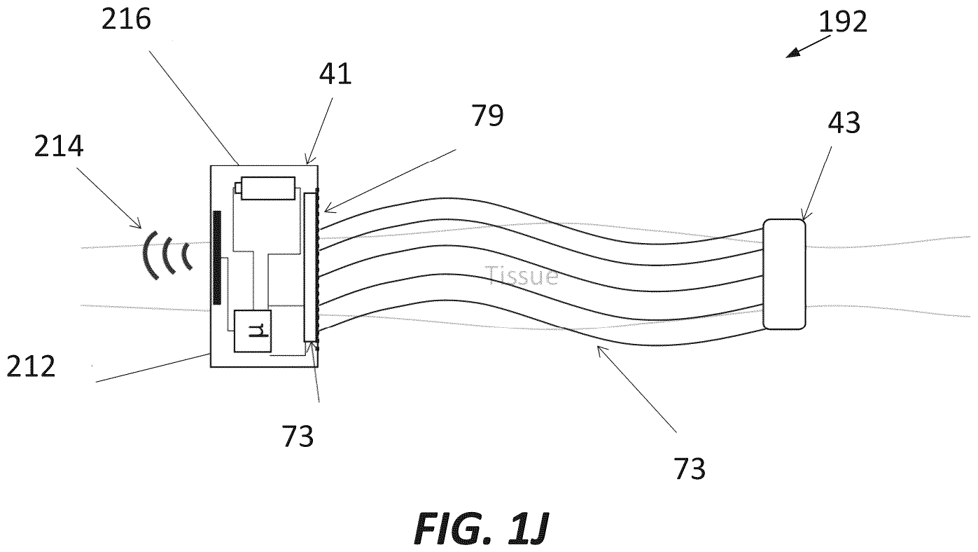

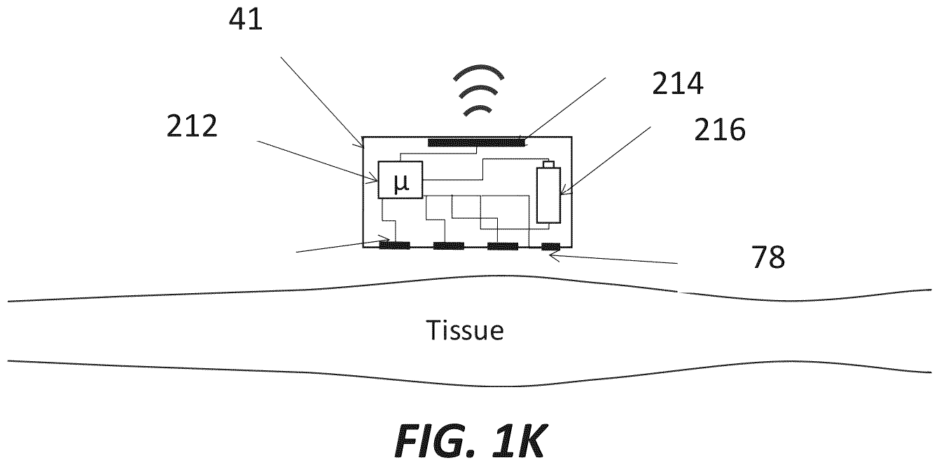

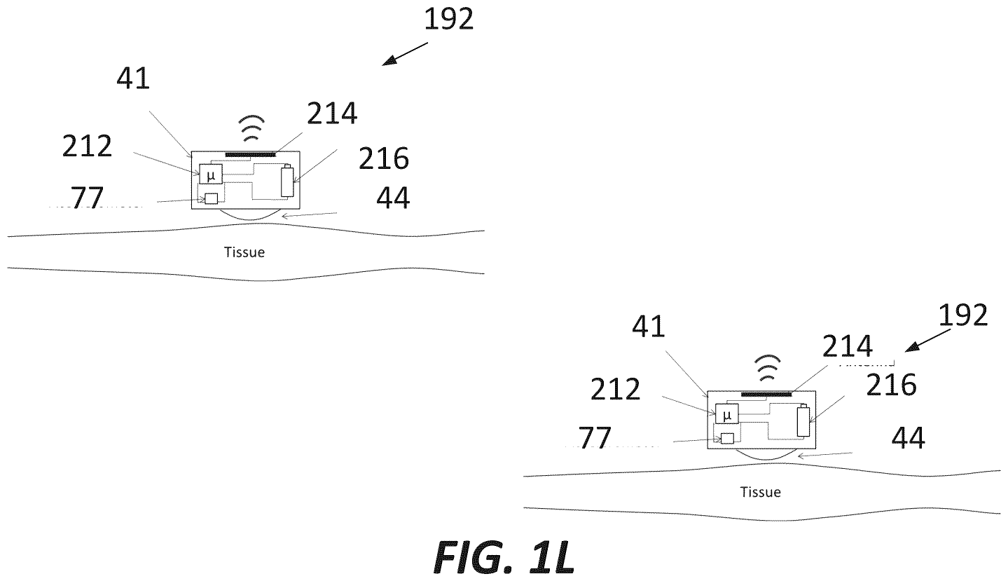

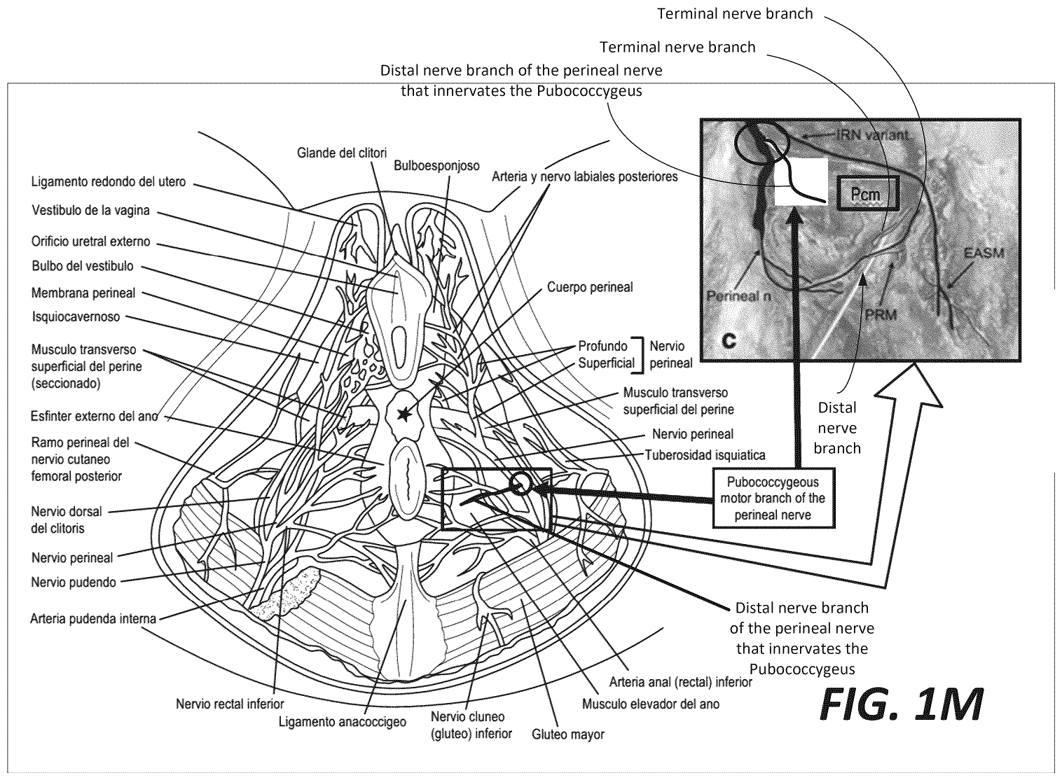

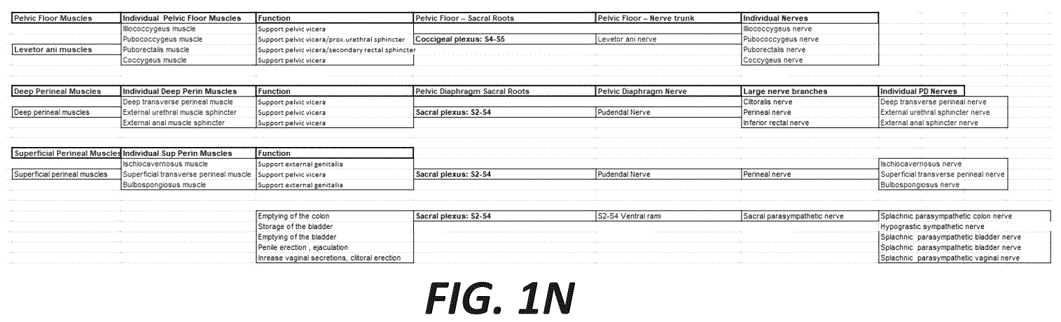

FIG. 1A schematically shows a patient 102 in accordance with illustrative embodiments. The patient 102 has just undergone a surgical procedure to implant one or more neuromodulation devices 100 and sensors 192. In various embodiments, the neuromodulation devices 100 are configured to stimulate one or more nerves. Nerves serve as conduits (e.g., electrical wires) within the body to carry signals from the central nervous system to peripheral anatomy (e.g. organs, muscles, hormone sources) and back. Nerve signals can either be autonomic (i.e. unconsciously controlled) or somatic (i.e. consciously controlled). In either application, nerves conduct a signal from a synapse on one end to a synapse on the other end.

These communication channels can deteriorate and/or be damaged over time due to disease, aging or injury. Damage can occur to the nerve (e.g. multiple sclerosis, vaginal delivery), the neuromuscular junction, or the effected organ (e.g. a muscle) at the end of a nerve. This damage can result in weakened or non-existent nerve signals passing from source to destination. Damage can also occur to the tissue that a nerve stimulates (e.g., a muscle or organ) affecting the ability of the tissue to receive and/or act on nerve signal inputs. Nervous system clinical conditions can result in 1) overactive nerve activity, 2) lost sensitivity, or the ability to sense weak source signals, and/or 3) poor conductance of signal, for example. These types of conditions can manifest as health issues, such as perceived pain, tingling sensations, and loss or weakening of muscle control.

Illustrative embodiments apply nerve stimulation and/or nerve recording devices 100, 192 to enhance, augment, and/or block nerve signal conduction. Some embodiments are provide nerve stimulation configured to promote healing and/or strengthening in either a subject nerve, a nerve connection to a receiving or transmitting tissue, a series of nerves, a network of nerves, tissue or organs that generate a nerve signal, and/or tissue or organs that receive a nerve signal (e.g. muscles). For discussion purposes, the nerves of the pelvic floor are referred to herein, however it should be understood that the devices and methods described herein are applicable to nerve treatments in general.

Nerve stimulation is the practice of applying exogenous energy to nerves. This energy is most often in the form of electrical stimulation. Nerve stimulation affects tissue and organs upstream and downstream from the nerve and/or the nerve itself. Various embodiments provide electrical energy to one or more nerves to achieve a clinical objective. Energy levels delivered can be at or above threshold, resulting in nerve depolarization and generation of an action potential (i.e. a signal conducting along the length of the nerve). Sub-threshold energy levels can also provide therapeutic benefits (e.g. rejuvenation of nerves, increasing muscle strength, decreasing pain, reducing muscle spasms, and improving blood circulation). In some embodiments, more than one type of stimulation is applied to a common nerve. The required activation threshold for each nerve differs. Generally, larger nerves (e.g. motor (efferent) nerves) have a higher activation threshold than smaller nerves.

Stimulation parameters are utilized to quantify the properties of the signal (e.g. electricity) applied to a nerve. Although general parameter settings are provided herein, it should be noted that selected stimulation parameters may vary from nerve to nerve, patient 102 to patient 102 and application to application. Optimal stimulation parameters are affected by one or more of electrode geometry, electrode material, the nerve target, and the physiology of the function that is modified, for example. The parameter settings are derived based on one or more of the type of treatment (nerve blocking, etc.), feedback from one or more probe stimulations, user settings, nerve characteristics (type, size), duration of treatment, feedback from one or more sensors, and nerve condition, for example.

Stimulation parameters are not always static. They can be altered (i.e. increased or decreased) within a treatment session and/or over a series of treatment sessions. For example, the applied current to a nerve can increase commensurate with nerve healing and/or strengthening to promote further nerve healing and/or strengthening. In other embodiments, the frequency of the stimulation pattern may be varied over time to modulate the therapy. In some embodiments, stimulation levels and or frequency are diminished over time to effectively wean a patient 102 from nerve stimulation therapy. Stimulation parameters of various embodiments for nerve stimulation may be mono-phasic (i.e. unipolar) while other forms may be biphasic (i.e. a stimulation of one polarity is followed by a stimulation of the opposite polarity to reset the condition of the nerve post stimulation). In some embodiments, nerve stimulation is tri-phasic in that a nerve is actively reset prior to stimulation by applying reverse polarization first. Discussion of a particular type of stimulation should be considered as disclosing all three of these forms.

Exemplary stimulation parameters include, but are not limited to, the following:
Stimulation Trigger: the causal event that initiates stimulation onset. Examples include but are not limited to endogenous nerve activity, direct patient 102 control, automation (e.g. ambulation program), analysis of the output of one or more sensors 192 (e.g. crossing a threshold), and a scripted stimulation schedule (i.e. timing).
Amplitude: The magnitude of the stimulation pulse in electrical potential difference (e.g. Volts) or electrical current.
Pulse frequency: Quantity of pulses per unit time for a series of 2 or more pulses.
Pulse duration: Duration of time that the amplitude is high during a single pulse.
Pulse Duty Cycle: The ratio of on-time to off-time during a pulse cycle period (a function of pulse duration and frequency).
Group Pulse Count: the quantity of stimulation pulses that occur in a burst of stimulation pulses (i.e. a group). This may last for a period of time known as burst duration.
Inter-pulse delay: The amount of time elapsed between the end of one pulse and the beginning of a subsequent pulse (related to pulse duration and duty cycle).
Stimulation Duty Cycle: The amount of time treating with pulses vs. not treating. Stimulation pulses can also be on demand rather than following a time schedule.
Stimulation period: Elapsed time between initiation of two sequential pulse groups.
Waveshape: The change in stimulation amplitude over time as a pulse is applied. (square, sinusoid, triangle, ramp, etc.)
Recovery time: The amount of time reverse stimulus is applied to provide active recovery in a biphasic waveform.
Recovery ratio: The ratio of stimulus duration to recovery duration. This can be applicable to biphasic and monophasic stimulations.
Phase: Mono vs. biphasic vs. triphasic

Various embodiments described herein relate to systems and methods for delivering electrical stimulation to nerves for therapeutic, rehabilitative, or restorative purposes. The stimulation may be applied to enhance, replace, or interfere with naturally occurring nerve signals, depending on the condition being treated and the desired physiological response. Electrical stimulation is delivered through one or more electrodes positioned proximate to a target nerve and is controlled by a stimulation controller 400, which may optionally be in communication with sensing components or signal processing modules.

In some embodiments, stimulation is used to modify or augment native nerve signals. This approach, referred to herein as signal modification stimulation, is particularly useful in patients with weakened, delayed, or incomplete neural activity. For example, in cases where a nerve produces low-amplitude signals or fails to sustain activation for the necessary duration, the system may detect the native signal and either amplify it, supplement it with a proportional stimulation, or replace it entirely with a preset stimulation pulse train. In some configurations, stimulation is prolonged beyond the duration of the natural signal to maintain the desired physiological effect. This modality may be employed, for instance, to enhance phrenic nerve signaling in order to restore or support respiratory function by stimulating diaphragm contraction.

Other embodiments include efferent nerve stimulation, wherein the stimulation is applied to induce downstream physiological effects. This form of stimulation may be employed when voluntary or endogenous neural control is diminished, absent, or requires supplementation. The stimulation signal is typically configured to exceed the amplitude of natural neural activity to ensure dominance over any residual signal conduction. Efferent stimulation can be used to activate muscle tissue, or direct organ function. Illustrative embodiments may stimulate the phrenic nerve to drive respiration, activation of gastric nerves to influence motility or secretions, and stimulation of the pelvic floor muscles to mitigate or prevent pelvic organ prolapse.

Afferent nerve stimulation may be applied to induce effects that travel upstream, toward the central nervous system. This modality may be configured as sub-threshold stimulation intended to interfere with abnormal or undesired sensory input—such as pain or overactive urge signals—or as threshold or supra-threshold stimulation that introduces novel sensory information to compensate for diminished afferent signaling. Afferent stimulation may be used to treat conditions such as overactive bladder, to promote physiological responses such as vasodilation via stimulation of renal or tibial nerves, or to substitute sensory feedback in cases where a patient's native sensory pathways are damaged or impaired.

In some embodiments, it may be beneficial to inhibit neural activity using blocking stimulation patterns. Blocking stimulation is configured to interfere with signal propagation within the nerve, thereby suppressing the transmission of undesired or pathological signals. This modality is useful for both afferent and efferent pathways and may be employed to reduce pain, suppress muscle spasms, or modulate autonomic responses. For example, blocking signals may be applied to the trigeminal or pelvic nerves to mitigate sensory discomfort, or to efferent nerves to inhibit spontaneous muscle contractions or dampen inflammatory responses. Blocking stimulation is typically delivered as sub-threshold or low-amplitude pulses with carefully configured timing and waveform characteristics to disrupt native signal conduction.

In some embodiments, restorative or repair stimulation is applied to promote the healing or strengthening of nerves that are damaged, underutilized, or experiencing degenerative changes. This type of stimulation may be delivered continuously or intermittently at or near the activation threshold to promote axonal integrity, neuromuscular junction health, and remyelination. Restorative stimulation may halt or reverse atrophy and encourage the reintegration of the nerve pathway into normal physiological function. For example, pelvic floor stimulation may be employed not only for immediate muscle activation but also to progressively strengthen the neuromuscular system over time.

Some embodiments apply multiple stimulation types through a single nerve interface. For instance, in the treatment of stress urinary incontinence, a perineal nerve may be stimulated with a first stimulation pattern to provide immediate efferent control of the pelvic floor muscles, while a second stimulation pattern may be simultaneously or sequentially applied to promote regenerative healing of the nerve and associated muscle tissue. Over the course of treatment, the parameters of the efferent stimulation may be reduced as muscle control improves, while the restorative stimulation is adjusted to accelerate repair and strengthen the neuromuscular system.

In any of the described stimulation modalities, the parameters of the applied signal may be programmed, modulated, or dynamically adapted based on therapeutic needs. Stimulation parameters may include waveform type (e.g., monophasic or biphasic; square, sinusoidal, triangular), amplitude (sub-threshold, threshold, or supra-threshold), pulse frequency, pulse duration, pulse duty cycle, inter-pulse delay, and the number of pulses per pulse train. Additional parameters may include the stimulation duty cycle (the ratio of active stimulation time to rest time), the stimulation period (time between pulse groups), and recovery-related features such as recovery time and recovery ratio. The system may further include means for triggering stimulation manually, on a schedule, or in response to sensed physiological signals. These parameters may be selected and adapted to optimize efficacy, safety, and patient 102 comfort, and may change over time as the condition of the patient 102 evolves.

Multiple treatments may be administered through a single nerve. This is accomplished, in some embodiments, by applying more than one stimulation pattern to a single nerve. In some embodiments, stress-induced urinary incontinence (SUI) may be treated by stimulating the perineal nerve with a biphasic stimulation pattern at a set frequency to arrest nerve generation (efferent stimulation) and a second lower frequency biphasic stimulation pattern to promote healing in the nerve, neuromuscular junction and the muscle (regeneration stimulation). This approach prevents urinary incontinence from the onset of treatment via efferent control while training the nerve to respond to a stimulus and strengthening the affected muscle to restore urinary flow control over time. It should be understood that as the ability for the nerve-muscle system to control urinary flow improves, the parameters for efferent stimulation and regeneration stimulation may change over time. In one example, efferent stimulation amplitude decreases over time (days) as the nerve-muscle system heals while regeneration stimulation amplitude increases over time to promote additional healing and strengthening.

FIG. 1B schematically shows a neuromodulation device 100 configured in accordance with illustrative embodiments. To that end, the neuromodulation device 100 has a main body 40 coupled with a movable arm 50 (also referred to as a movable jaw 50). The arm 50 may be hingedly coupled with the main body 40 (e.g., such that the arm 50 pivots relative to the main body 40).

In various embodiments, the main body 40 may include a housing 48. The housing 48 may also encapsulate at least a portion of the movable arm 50. To that end, the housing 48 may be formed from a resilient or deformable material. The main body 40 and the movable arm 50 (or the portion of the housing 48 surrounding the movable arm 50 and the main body 40) may define a chamber 101 configured to receive a nerve 200 and a channel 102 leading to the chamber 101. In some embodiments, the chamber 101 may be defined by one or more arms 50. The chamber 101 includes at least one electrode 104 to stimulate the nerve 200.

The housing 48 encapsulates a package 46 of the main body 40. The housing 48 includes a buffer layer 49. As known by those of skill in the art, the package 46 encapsulates electronics and semiconductor material within. For example, the package 46 may include different types of circuitry, including stimulator(s), sensor(s), communication(s), and/or power circuitry. In various embodiments, the package 46 may be formed from a substantially rigid material, such as titanium, stainless steel, glass, ceramic, alumina, zirconium, plastic, and/or other generally acceptable hermetic package material. In various embodiments, the electronic package and the nerve/attachment electrode are unitary. In various embodiments, instead of a feedthrough pin extending from the hermetically sealed housing that is welded to the electrode, the feedthrough pin forms the electrode. In such embodiments, the electrode is unwelded (i.e., has no welded joints connecting it to the feedthrough pin). Instead, the feedthrough pin forms the electrode. When the feedthrough pin is embedded in the movable arm (e.g., silicone) it operates as a reinforcement for the deflecting arm.

The package 46 forms a hermetic seal around the internal electronic circuitry. The electronic circuitry is preferably loaded into the hermetic enclosure 46 and sealed in an inert oxygen and water-limited environment. After the hermetic seal is formed, two advantages are provided. First, the hermetic seal ensures that no additional liquid is introduced into the electronic package 46, potentially causing electrical failure. Second, the hermetic seal ensures that the non-biocompatible materials from which the internal electronics are formed do not leach into the body.

Preferably, the package 46 is formed from a glass or ceramic material, which provide reduced interference to radio frequencies used for wireless power and wireless communication relative to other commonly used package 46 materials. The package 46 (also referred to as the hermetic enclosure 46) may be formed from glass wafers directly bonded to one another. Alternatively, the package 46 may be formed from zirconia ceramic with brazed feedthroughs. In various embodiments, the package 46 may be sealed using laser welding titanium surfaces brazed to the zirconia.

The buffer layer 49 covers the package 46 and provides a second biocompatible layer and suitable soft surface within the human body and continuity to portion contacting the nerve 200. As mentioned previously, the arm 50 may include the overmolded housing 46 formed of a softer material (e.g., durometer 30-50 Shore A).

The buffer layer 49 may be formed from a material configured to conform within its resting position in the body. Although the arm 50 and the package 46 may be encapsulated by the housing 46, the buffer layer 49 does not cover the electrodes 104. Accordingly, the housing 48 may have openings for the electrodes 104 and/or an EMG 42. Additionally, vias 45 may be formed through the housing. In various embodiments, the buffer layer 49 can encapsulate between about 20% and about 99% of the device.

Vias 45 may extend through the package 46 and/or the housing 48 (including buffer layer 49). Electrodes 104 and other external electrical connections may be connected to the metalized vias 45. The metalized vias may be formed using platinum feedthrough wires, among other things. The package 46 and the buffer layer 49 may be joined using RTV biocompatible silicone.

In FIG. 1B, the arm 50 is depicted in a closed position or a substantially closed position (collectively referred to as the closed position of the device), in which the size of the channel 102 is sufficiently small so that the nerve 200 cannot pass through the channel 102. The arm is movable from the closed position to an open position in which the size of the channel 102 is sufficiently large so that the nerve 200 can pass through the channel 102. In some embodiments, the open position is sufficiently large such that the nerve 200 can be pass through the channel with a reduction in diameter of less than about 30% to about 50% (e.g., by stretching and/or squeezing the nerve through the channel). Various embodiments may be configured to receive a variety of nerve sizes, as discussed further below. In various embodiments, the arm 50 may be biased (e.g., by the use of a biased interior member 51) towards the closed position. A medical practitioner may transition the arm 50 to the open position by applying a force to the arm 50 (e.g., by pulling on a grippable extension 52) or by pressing the arm 50 with the nerve 200 itself. The nerve 200 may then pass through the channel 102 into or out of the chamber 101.

FIG. 1C schematically shows a perspective view of alternative embodiment of the device 100 (with details of the chamber 101, such as the electrode 104 omitted). The channel 112 has a particular travel path 113 (also referred to as a central axis 113) for the nerve 200. For example, as shown in FIG. 1C, the central axis 113 is non-linear. The central axis 113 may be defined by the arms 50.

In addition to the central axis 113, the device 100 has a longitudinal axis 112 that is orthogonal to the central axis 113 at any given point. During nerve implantation procedure, the nerve 200 is generally parallel to the longitudinal axis 112 as it travels along the central axis 113. Two different longitudinal axes 112A and 112B are shown for two different points along the central axis.

FIG. 1D schematically shows another embodiment of the neuromodulation device 100 in accordance with illustrative embodiments. FIG. 1D shows four different views of a neuromodulate device 100. The hermetically sealed main body 40 is largely omitted. However, a feedthrough conductor 80 that extends from the interior of the hermetically sealed main body 40 is shown. The feedthrough conductor 80 extends through the package 46 and the buffer layer 49. The feedthrough conductor 80 then forms the electrode 104 within the chamber 101, which is defined by the movable arm 50.

The movable arm 50 is shown biased towards a closed position. Although referred to as the “closed position,” some embodiments may have the gap 76. Therefore, it is not necessary that the movable arm 50 entirely close the gap 76 in the closed position. The closed position 76 is used to refer to the position of the channel 102 when the channel is narrow to retain the nerve. As the nerve passes through the channel 102, the gap 76 increases (e.g., because the arm 50 moves and/or deforms) and the movable arm 50 transitions towards the open position. To help facilitate the opening of the channel, the movable arm 50 may include one or more bending points 81 formed from a material configured to bend, in order to accommodate the nerve 200. It should be understood that the gap 76 does not have to be fully closed to be biased towards the closed position. In a similar manner, the gap 76 does not have to be fully open to transition towards the open position.

FIGS. 1E-1L schematically show various sensors 192 in accordance with illustrative embodiments. In some embodiments sensors 192 are implanted within the body. In some embodiments, the sensors 192 are within a cavity (e.g. mouth, vagina) or digestive tract of a patient. In some embodiments, sensors 192 are external to the body (e.g. worn in garments, adhered to the skin, located in a watch, etc.).

Implantable sensors 192 can be individual, combination or integrated into another system component (e.g. nerve stimulation chamber, controller 400, etc.). Some embodiments of an implantable sensor 192 are powered by a battery. Some embodiments of an implantable sensor 192 are powered by electromagnetic energy (e.g. RF energy) whereby the electromagnetic energy induces current into an electrical circuit in electrical communication the sensor. In some embodiments, the sensor 192 is read (e.g. voltage measured, current measured) by a circuit or processor and that information (e.g. a value) is communicated to a controller 400 by wired or wireless means. In one exemplary embodiment, an implantable pressure sensor 192 is powered by RF energy whereby upon receiving said energy, the pressure sensor 192 is read and the measurement value is transmitted to a receiving device. This is analogous to an RFID device 100 that is powered by RF energy and reports back information. It should be understood that all of the implantable sensors 192 presented herein can be powered by battery, wirelessly, or both. In some embodiments, a battery within the implantable component (e.g. sensor) is recharged from an implantable conductive coil when electromagnetic energy induces current in a coil within the implantable component.

Some embodiments of an implantable sensor 192 include a housing 41 to protect electrical and other elements within the sensor 192 from exposure to tissue and liquids. In other embodiments, electrical components are potted in one or more materials (e.g. epoxy or silicone) and there is no housing component. It should be understood that for the implantable sensor 192 types presented herein, an enclosure can be optional.

In various embodiments, any of the sensors 192 described herein may include one or more integrated components such as a local processor 212, an antenna 214 (for wireless communication and/or power transfer), and/or an internal power source 216 such as a battery or capacitor. For example, a sensor 192 may be configured with onboard processing to perform signal filtering or feature extraction prior to transmitting data to a principal controller. Similarly, one or more sensors may include antennas configured to receive power via inductive coupling or to wirelessly transmit sensor data to a neuromodulation device or external receiver. In some configurations, sensors may also be fully self-powered using miniature energy storage elements. However, these additional components are not required in all embodiments. In other implementations, one or more of the processor, antenna, or power source may be omitted, with the sensor instead relying on hardwired connections, remote processing, or passive telemetry systems for functionality. The sensor architecture is thus modular and may be adapted based on space constraints, energy demands, communication requirements, or clinical objectives.

FIG. 1E depicts an implantable electromyogram sensor 192 with two electrodes. The sensor 192 depicted is placed near the muscle, however other embodiments utilize electrical leads that extend from the sensor 192 to the muscle. The device 100 measures a voltage across the electrodes that is proportional to muscle activity. As shown, there is an enclosure housing electrical circuitry and two leads that extend to sensing ends (electrodes).

The tethered electrode connection enables the electrodes to maintain their location with respect to the muscle by moving relative to the housing and each other during muscle contraction and relaxation. In another embodiment (not shown), one electrode is within the sensor 192 housing 41 and a second electrode is tethered to the housing. In some embodiments, electrode interface with the muscle with barbs 43 (shown). In other embodiments, electrodes include materials that promote tissue ingrowth (e.g. open cell foam) to anchor the electrode in place. Any of the various sensors 192 described herein can be tethered in a similar manner.

Electrodes may be formed from electrically conductive materials (e.g. metals (platinum, gold, silver, stainless steel), electrically conductive polymers (e.g. polypyrrole, PEDOT-PSS), and/or carbon (e.g. graphene, carbon nanotubes).

Some embodiments may include an Electroneurogram (ENG) sensor 192. The ENG sensor 192 is configured to detect an ENG signal from a target nerve that innervates the target muscle. In various embodiments, depending on the condition being treated, the ENG signal may be obtained from a variety of nerves including but not limited to the following:
OAB: Pelvic sensory nerve carrying mechanoceptive information from the bladder informing vesical pressure. Pelvic sensory-motor perineal nerve, pudendal nerve, perineal nerve branches such as the pubbococygeous nerve.
FI: Sensory and sympathetic nerves in the gut including vagal and pelvic afferent nerves. Sensory nerves in the gut wall. Sensory-motor nerves: puborectalis muscle and branches of the perineal nerve innervating the internal and external anal sphincter, inferior rectal nerve, pudendal nerve.
SUI: Sensory-motor nerves from the pubococcygeous muscle, sensory pelvic nerve, branches of the perineal and pudendal nerves. Branches of the levator ani nerves.
POP: Branches of the levator ani nerves, perineal nerves, inferior rectal nerves, coccygeus nerve, iliococcygeus nerve, perineal nerve, inferior rectal nerve, pudendal nerve, pubococcygeus nerve, and puborectalis nerve.

FIG. 1F schematically shows a nerve conduction velocity (NCV) sensor arrangement in accordance with illustrative embodiments. The sensors 192 in some embodiments may be the neuromodulation devices 100 (e.g., satellite devices 100B). In some embodiments, nerve conduction velocity is measured by stimulating a nerve with a stimulation electrode and recording a nerve with a recording electrode, the electrodes spaced a known distance apart and located within a common chamber. A processor connected to the electrodes quantifies the signal transit time between stimulation and recorded signal in the nerve. Subsequently, the processor divides the distance between the electrodes by the signal transit time to derive the nerve conduction velocity. In some embodiments, the nerve stimulation and signal recording are performed by separate devices that communicate timing information to each other using wired or wireless means.

Nerve conduction velocity provides a quantitative means to determining the health of a nerve. Longer signal transit times indicate a nerve with compromised performance (e.g. Multiple sclerosis, diabetic neuropathy). The stimulation treatment applied to a nerve in poor health can inform the patient's body that the nerve is still in use, thereby arresting degradative processes and promoting healing processes. This can lead repair of the nerve condition and improvement in NCV. In various embodiments, depending on the condition being treated, the NCV signal may be obtained from various nerves. In the area of pelvic health, the following exemplary nerves may be treated:
OAB: Pelvic sensory nerve carrying mechanoceptive information from the bladder informing vesical pressure. Pelvic sensory-motor perineal nerve, pudendal nerve, perineal nerve branches such as the pubbococygeous nerve.
FI: Sensory and sympathetic nerves in the gut including vagal and pelvic afferent nerves. Sensory nerves in the gut wall. Sensory-motor nerves: puborectalis muscle and branches of the perineal nerve innervating the internal and external anal sphincter, inferior rectal nerve, pudendal nerve.
SUI: Sensory-motor nerves from the pubococcygeous muscle, sensory pelvic nerve, branches of the perineal and pudendal nerves. Branches of the levator ani nerves.
POP: Branches of the levator ani nerves, including coccygeus nerve, iliococcygeus nerve, and pubococcygeus nerves, perineal nerve, inferior rectal nerve, pudendal nerve, and puborectalis nerve.

FIG. 1G schematically shows an implantable pressure sensor 192 in accordance with illustrative embodiments. The pressure sensor may have a pressure sensing element 73, as well as thin metallic membrane 75. Pressure within a cavity (e.g. a bladder) induces pressure on the cavity walls and strain in the cavity walls. Hence, pressure information can be obtained from either pressure sensor 192 configured to detect a pressure signal or a strain sensor. Pressure measurements may be utilized as input into a nerve stimulation and/or recording system. Exemplary embodiments may measure the pressure and/or strain signal from a variety of anatomical locations including the following:
Bladder/vesical pressure: pressure sensor 192 in or near the lumen of the bladder.
Urethral pressure sensor: pressure sensor 192 in or near the lumen of the urethra,
Rectal pressure sensor 192 in the lumen of the rectum.

FIG. 1J schematically shows an implantable tissue strain sensor 192 in accordance with illustrative embodiments. Strain pressure sensors 192 on the external surface of organs or cavities to detect distention under pressure sensor 192. Strain sensors 192 can be also placed on ligaments in the pelvic floor to provide feedback of strain on the structural components of the pelvic floor.

FIG. 1H schematically shows an implantable movement sensor 192 in accordance with illustrative embodiments Movement sensor: In some embodiments, a nerve stimulation system utilizes data from a patient 102 motion sensor 192 (e.g. an accelerometer) as input into the nerve stimulation control. In one exemplary embodiment, when patient 102 motion (e.g. walking) is detected, the nerve stimulator initiates or increases nerve stimulation levels to nerves controlling urethra sphincter muscles to prevent bladder leakage. Conversely, when the nerve stimulation system detects that patient 102 motion has ceased, in some embodiments, the nerve stimulation level is either decreased or stopped entirely.

FIG. 1I schematically shows two implantable pressure sensors 192 functioning as a relative movement sensor 192 in accordance with illustrative embodiments. The relative movement sensor similar to the movement sensor but includes a set of two sensors. Motion of structures within the body can inform a nerve stimulation system of physiologic conditions. Exemplary relative motion events that a nerve stimulation system can sense include:
Relative movement of pelvic structures to inform neural responses to natural movement.
Relative position of opposing sides of the stomach to quantify stomach distention and detect peristaltic activity.
Relative position of organ bodies to inform timing of stimulation regimes.
Relative stability of structures to assess disease progression, stimulation protocol adjustments, and fatigue.

In some embodiments, a relative motion sensor 192 utilizes ultrasonic waves to detect the relative movement between two components. In one embodiment, a first component sends out an ultrasonic signal that reflects off a second component and returns to the first component where it is sensed. The distance between the two components is derived by multiplying the elapsed time by the speed of ultrasound within tissue (1540 m/s) and dividing by 2. In another embodiment, a first component transmits an ultrasonic signal and second component receives an ultrasonic signal. The two components are synchronized within the system such that a controller can determine the elapsed time between the signal being sent and the signal being received. The distance between the components is determined by multiplying the elapsed time by the speed of ultrasound within tissue.

In some embodiments, a nerve stimulation/recording device 100 receives information from a User or clinician through a wired or wireless connection. In some embodiments, the received information directly informs the nerve stimulation device 100 to stimulate one or more nerves (e.g. like an on/off switch). In some embodiments, the received information provides input into an algorithm that calculates one or more of 1) whether or not to stimulate one or more nerves, 2) the level of stimulation, and 3) the type of stimulation, and 4) duration of stimulation. For example, the patient 102/User feedback may include information about:
OAB: frequency, incidence, or magnitude of urinary urgency, frequency, incidence, or magnitude of urination, frequency, incidence, or magnitude of nocturia, frequency of urge incontinence, and/or pain or sensation from the stimulation.
SUI: frequency, incidence, or magnitude of urinary leakage and associated activity (e.g., sexual activity, lifting weights, sedentary, etc.), and/or pain or sensation from the stimulation.
FI: frequency, incidence, or magnitude of fecal leakage, frequency of passive incontinence, frequency of urgency and/or pain or sensation from the stimulation.
Pelvic organ prolapse: frequency, incidence, or magnitude of bulging sensation, vaginal symptoms, and/or pain or sensation from the stimulation.
Erectile dysfunction: erection start, erection stop.
Pain level: Patients may report to a nerve stimulation device 100 their level of sensed pain from a clinical condition (not the nerve stimulation), enabling a nerve stimulation system to titrate the level of stimulation required to block pain signals from reaching the brain.

FIG. 1K schematically shows an implantable electrochemical sensor 192 in accordance with illustrative embodiments In some embodiments, an implantable chemical sensor 192 is utilized by a nerve stimulation system to detect the level of specific chemicals (e.g. hormones, saliva, mucous, endocrines, proteins) at a location within the body. This information is then utilized as one or more of feedback and triggering information for a nerve stimulation system. In one embodiment, a chemical sensor 192 is an implantable electrochemical sensor. In one embodiment, a chemical sensor 192 is a carbon nanotube chemical sensor. The electrochemical sensor 192 may include a series of electrodes 78.

FIG. 1M schematically shows an anatomical drawings of nerves that neuromodulation devices may couple to in accordance with illustrative embodiments. A nerve branches from the central nervous system (CNS) towards a muscle by first exiting the spinal cord through spinal roots. These roots converge to form peripheral nerves that traverse the body. As the nerve reaches the target muscle, it divides into smaller branches, ultimately connecting at neuromuscular junctions. This branching allows the nerve to transmit motor signals from the CNS, facilitating muscle contraction and controlling voluntary movements.

The anatomy of a nerve extending from the central nervous system (CNS) to a target muscle is a complex process that involves several distinct stages. Once outside the spinal column, the spinal nerves branch out and may combine with axons from other spinal nerves to form networks called plexuses (e.g., brachial plexus for the arms, lumbar plexus for the legs). From these plexuses, numerous peripheral nerves emerge, directed towards specific areas of the body.

As the peripheral nerves approach their target muscles (towards a distal end of the nerve), they branch into smaller motor nerves. These motor nerves carry the impulses necessary for muscle contraction. At the target muscle, the nerve fibers terminate in specialized structures called neuromuscular junctions. Here, the axon terminal of a motor neuron releases neurotransmitters (mainly acetylcholine) across a small gap, the synaptic cleft, which binds to receptors on the muscle fiber's membrane (sarcolemma).

What is not shown well in many anatomical drawings in the art are the various sub-branches towards the distal end of the nerve (i.e., near the neuromuscular junction). This is because the number of branches a nerve can have varies widely depending on the specific nerve and its function. Nerves can branch off into many smaller nerves as they extend from the central nervous system (CNS) to their target organs or tissues. It is difficult to illustrate all of the anatomical nerve structures when including sub-branches, which have largely been ignored by the prior art as a source of treatment.

Advantageously, various embodiments couple one or more neuromodulation devices to the small distal nerve branches that innervate the target muscles discussed herein. However, illustrative embodiments may additionally or alternatively couple one or more neuromodulation devices anywhere along a given nerve (e.g., not just at or near the distal nerve branch).

When a large nerve branches, each branch may carry a different number of fascicles. For instance, when the sciatic nerve divides into the common peroneal and tibial nerves, each branch carries a subset of the fascicles from the main nerve. The division isn't necessarily equal, and the number of fascicles in each branch depends on the specific functions and size of the area each branch innervates. As these branches subdivide further to innervate specific muscles or skin areas, the number of fascicles in each smaller branch continues to decrease. Various embodiments couple one or more neuromodulation devices to a distal nerve branch that innervates a pelvic floor muscle, where the distal nerve branch has as few as 1-3 fascicles. In some embodiments, the distal nerve branch may be a terminal nerve branch. However, as mentioned above, illustrative embodiments may couple one or more neuromodulation devices to a larger portion of the nerve.

In addition to, or alternatively to, identifying distal nerve branches by their fascicle count, it is also possible to define the distal nerve branches by their location along the nerve. A nerve originates at a soma and has an axon extending from the soma to an axon terminal. The distal nerve branch that innervates a pelvic floor muscle may be at the last ⅓ or last ¼ of the length of the nerve (e.g., near the distal end closest to the muscle.). Various embodiments couple one or more neuromodulation devices to a distal nerve branch at a point that is along the last ⅓rd or ¼th of the length of the nerve.

FIG. 1M schematically shows a detailed view box that illustrates the distal nerve branches. As discussed previously, these distal nerve branches are generally not shown in anatomical drawings due to the large number, and do not have well defined anatomical names. In the past, the inventors have referred to the distal nerve branches by the anatomical name of the major nerve from which they branch. However, to provide further clarity, illustrative embodiments may refer to the distal nerve branch by the name of the nerve from which it branches and also the muscle which it innervates. However, it is understood that one or more particular distal nerve branches from a larger nerve may innervate a particular muscle. Furthermore, various embodiments stimulate with nerves approaching from the right and left sides of the body such that stimulation of a nerve and its contralateral nerve may achieve a desired clinical effect. In various embodiments, one or more of the identified distal nerve branches may be stimulated.

In particular, FIG. 1M also shows a zoomed-in detailed view of a distal portion of a perineal nerve branch that innervates the pubococcgyeus muscle. The distal nerve branches (including terminal branches) are shown.

Various embodiments provide a closed-loop stimulation of nerves that innervate the pelvic floor muscles to treat a variety of conditions including SUI, OAB, FI, and/or pelvic organ prolapse. The closed-loop stimulation continuously monitors specific physiological signals and provides a therapeutic stimulus in real time based on this feedback.

Various embodiments may couple and stimulate nerves to treat, among other things: female sexual dysfunction, male erectile dysfunction, fecal incontinence, overactive bladder/urge incontinence including, pain management including chronic pelvic pain, headaches, somatic (i.e. muscle, limbs) and visceral either by activating mechanoreceptive or proprioceptive nerve fibers that compete with nocioceptive signals or by directly blocking the signals of pain nerve fibers, blood pressure management, sleep apnea, stimulation for nausea, stimulation for tremors, and/or stimulation of the vagus nerve, sacral roots and ventral roots for various applications. Various embodiments may stimulate various nerves in accordance with the stimulation parameters provided herein (e.g., for OAB, SUI, FI, and/or POP) and for other treatments in accordance with the same or different parameters (e.g., as those used for OAB, SUI, FI, and/or POP).

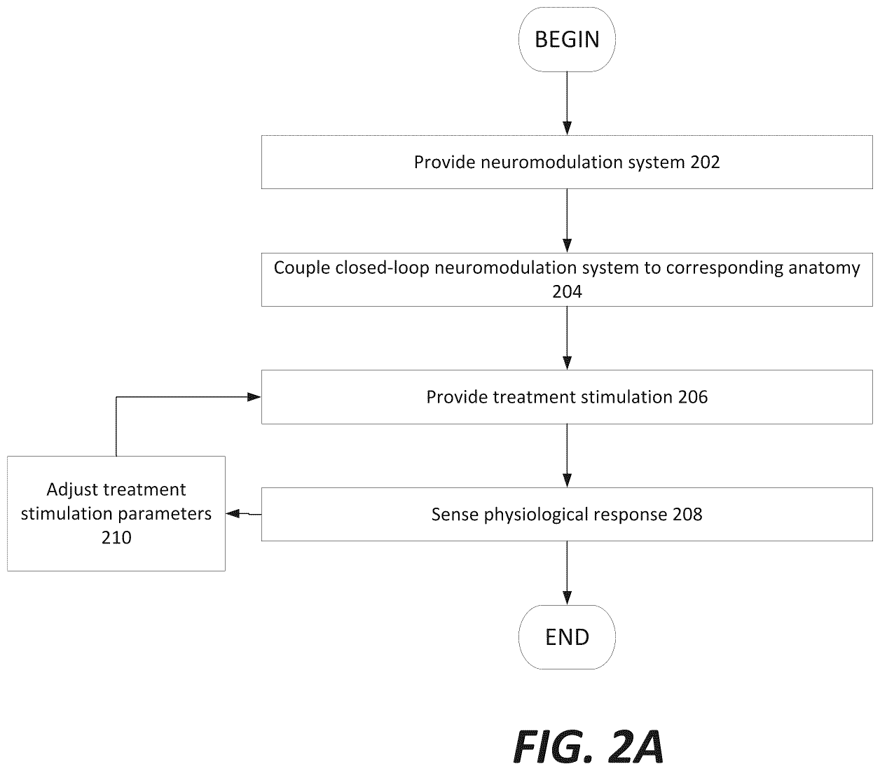

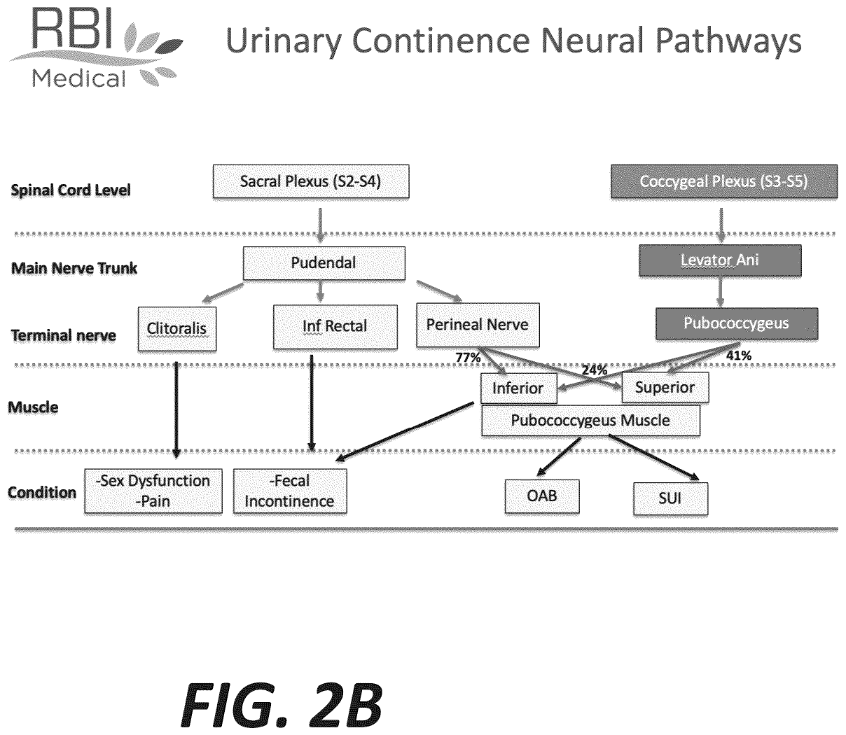

FIG. 2A shows a process for closed-loop neuromodulation in accordance with illustrative embodiments. It should be noted that this process is simplified from a longer process that normally would be used. Accordingly, the process likely has many steps that those skilled in the art likely would use. In addition, some of the steps may be performed in a different order than that shown. Additionally, or alternatively, some of the steps may be performed at the same time. Those skilled in the art therefore can modify the process as appropriate.

In various embodiments, a process of using the system for closed-loop stimulation begins at step 202, which provides a neuromodulation system. The system includes one or more sensors 192 configured to sense a signal from a patient, one or more neuromodulation devices (also referred to as a stimulators) configured to stimulate a target tissue (e.g. a nerve), and a controller configured to provide the therapeutic output as a function of the sensed signal.

The neuromodulation device 100 may be any of the devices discussed herein, and may include the primary neuromodulation device 100 with the satellite neuromodulation device(s) discussed further below.

As will be discussed further below, the controller is configured to receive data from the one or more sensors 192 and uses the sensed data to modulate a therapeutic dosage. Various embodiments may refer to receiving data from the sensor, but it should be understood that reference to the sensor 192 in the singular also includes data receiving from one or more sensors. In some embodiments, the controller and/or the sensor 192 may be integrated into one or more of the neuromodulation devices. Additionally, or alternatively, the controller and/or the sensor 192 may be separate from the one or more neuromodulation devices. In some embodiments, the controller and/or sensor 192 are external to the patient's body.

At step 204, the closed-loop neuromodulation system is coupled to corresponding anatomy. In particular, the one or more neuromodulation devices are coupled to one or more corresponding nerves. In one embodiment, the target nerve is positioned into a stimulation chamber of a neuromodulation device. In some embodiments, the device 100 includes at least one movable arm that opens to permit passage of the nerve into the stimulation chamber. The arm closes to prevent or hinder the nerve from being dislodged from the stimulation chamber without overly compressing the nerve. In some embodiments, the movable arm is configured to retain nerves of a variety of sizes within the chamber (e.g., the chamber accommodates nerves of ranges of 0.3 mm-4 mm). In various embodiments, the arm is biased towards a closed or substantially closed position.

Additionally, the device 100 may be configured to retain nerves of different sizes in the chamber and in contact with one or more electrodes while applying an atraumatic retention force on the nerve. The channel similarly is configured to allow passage of the nerve therethrough. In some embodiments, the channel can yield to pressure from inserting nerve, such that the channel opens or changes shape. The necessary pressure to open or deform the channel is configured to be less than a pressure that may damage the nerve. Additionally, or alternatively, the nerve is stretched in length causing its cross-sectional diameter to narrow to a point that it can pass through the channel. In some embodiments, both the channel and the nerve deform to some degree to permit the nerve to pass through the channel. Details of illustrative embodiments are discussed below.

As discussed previously, various embodiments further selectively couple to and stimulate the peripheral nerve in areas near the distal end of the nerve (e.g., the last ¼th or ⅓rd length of the nerve, or where there are a small number of fascicles 1-3). This allows the use of a compact stimulation device 100 to deliver lower power stimulation directed at a specific nerve target without the risks of a potentially damaging, time intensive and/or traumatic surgical procedure. Prior art electrode arrays disadvantageously do not provide specific enough stimulation to the targeted nerve often resulting in unintended side effects (e.g. non-target nerve stimulation). Similarly, nerve cuffs disadvantageously are limited by pre-defined sizes, which often lead to suboptimal nerve electrical coupling when a cuff is not properly sized. Various embodiments disclosed herein advantageously provide greater assurance of electrical coupling to a target nerve for delivery of a selective stimulation signal and are placed with a simple implantation procedure.

Furthermore, various embodiments include a neuromodulation device 100 having a channel defined by a plurality of jaws or arms that are movable and/or deformable. The dimensions of the channel are adjustable to allow for entry of nerves of various sizes into the chamber, while also for securing the nerve within the chamber with electrode contact. Additionally, other sensors 192 may be coupled to relevant anatomy (e.g., EMG may be coupled to the corresponding muscle, etc.).

Sensors utilized for system triggering and/or feedback are coupled to relevant anatomy and may be located within or external to the patient's body. In some embodiments, the triggering and feedback sensors 192 are independent sensors. In some embodiments, the EMG sensor 192 placed over a specific muscle is utilized to trigger a nerve stimulator that is coupled to a nerve that stimulates the same muscle. When a patient 102 consciously tries to use the muscle, the output of the EMG sensor 192 changes which is detected as a muscular contraction by the nerve stimulation controller. The controller then stimulates the nerve that innervates the subject muscle to assist in muscle contraction. In some embodiments, the nerve stimulation system ends stimulation when the same or other sensors 192 detect that muscular contraction is complete (e.g. relative motion sensors 192 indicate that an orifice is closed). In another embodiment, nerve stimulation ends after a duration of muscular contraction time (e.g. voiding the bladder requires diaphragm and abdominal muscle contraction for an amount of time).

A nerve stimulation has a waveform, frequency and amplitude. Nerve stimulations can be applied to a nerve at different times. In some embodiments, a nerve stimulation is applied based on feedback from a sensor. In some embodiments, a nerve stimulator includes a sensing capability. In some embodiments, a nerve stimulator determines the nerve stimulation parameters to use based on the shape, magnitude and/or timing of one or more sensed parameters. In some embodiments, the sensed measurements of a first nerve stimulator are utilized as feedback for a second nerve stimulator.

At step 206, a treatment stimulation is provided. Stimulation signals may vary, depending on the condition to be treated, a different signal may be applied. When multiple stimulator devices are coupled to multiple different nerves, the stimulation parameters (e.g. timing, amplitude, frequency and waveform) provided from each stimulator may vary. In this way, a collection of stimulator devices can orchestrate tissue (e.g. nerve, muscle, organ) responses in time. In one example, a collection of stimulator devices coupled with ambulatory skeletal muscles (e.g. gluteus maximus, gastrocnemius, soleus, quadriceps) can direct a pattern of muscular contractions and relaxations that enable a patient 102 to walk. In another embodiment, a collection of stimulator devices can enable urination while inhibiting defecation.

Various embodiments provide a closed-loop stimulation. A test or probe stimulation is generated by the nerve stimulator, applied to a target nerve, and feedback is detected via a sensor. The feedback is used to adjust subsequent stimulations. For example, if no feedback is detected, one or more stimulation parameters (e.g. duration, amplitude, waveform, frequency) are altered and the probe stimulation is repeated.

In some embodiments, the system conducts probe stimulations at defined intervals to evaluate changes in motor response thresholds. For example, the system may apply a low-intensity stimulation and measure the response (e.g., evoked EMG amplitude or latency). If the stimulation elicits a motor response at a lower threshold than a previous session, the system interprets this as a sign of increased nerve or muscle responsiveness—and may update future therapy parameters accordingly.

Thus, in various embodiments, the process involves the application of at least one initialization probe stimulation to identify and characterize the motor and proprioceptive nerve branches, after which therapeutic stimulation is delivered and dynamically modulated based on real-time physiological feedback. In some embodiments, sensed feedback from a non-probe stimulation is utilized as feedback to the system to inform parameter settings for one or more subsequent stimulations.

Stimulation patterns can also vary based at least in part on nerve size, patient 102 sensitivity, duration of treatment among other parameters.

Some exemplary probe stimulation patterns for specific treatments are presented below:
1) An “OAB probe stimulation” to evoke a detectable physiological response from muscles related to OAB syndrome, and
2) An “SUI probe stimulation” to evoke a detectable physiological response from muscles related to SUI syndrome,
3) A “FI probe stimulation” to evoke a detectable physiological response from muscles related to SUI syndrome, AND/OR
4) A “POP probe stimulation” to evoke a detectable physiological response from muscles related to SUI syndrome.

In some embodiments, the probe stimulations are configured prior to sending the treatment stimulation. In various embodiments, the probe stimulations may be for OAB, SI, FI, and/or POP and/or a combination thereof. In some embodiments, the probe stimulation uses the same parameter settings as a treatment stimulation. Indeed, in various embodiments, the probe stimulation may be the beginning of the treatment therapy described herein, or it may be different. The probe stimulation is used to evoke a tissue response that is sensed and characterized (e.g. quantified). The tissue response may be characterized for one or more of response magnitude, response duration, and response onset delay. These characterizations are used by the nerve stimulation controller as feedback for updating subsequent treatment (e.g., subsequent stimulation parameters).

In another example of closed-loop feedback, a nerve stimulation system includes of a controller, energy source, electrode, and electrochemical sensor. The electrochemical sensor 192 is utilized by the system to measure the level of a specific chemical (e.g. blood sugar) in the body. When the blood sugar level rises above a threshold value (e.g. >150 mg/dL), the controller 400 begins applying an appropriate stimulation pattern to the vagus nerve to stimulate the pancreas to secrete additional insulin. The system continues to monitor blood sugar levels (feedback) and ceases vagus nerve stimulation when indicated blood sugar levels reach a normal range (<130 mg/dL).

Stimulation parameter specifications and tissue response data may be stored in a database, memory, or other storage medium for use in subsequent steps. Among other locations, such a storage device 100 may be part of the nerve stimulation device 100 100 itself, part of the signal generator, part of another device 100 near the patient 102 (e.g., a patient 102 mobile device), across a local area network (e.g., an enterprise network), or across a larger wide area network (e.g., the Internet).

In some embodiments, a nerve stimulation system applies at least one “probe stimulation” to the target tissue to characterize the tissue response and provide input into the nerve stimulator settings in order to modulate the stimulation of the appropriate nerve. In one embodiment, one of the coccygeal plexus somatic nerves, such as the perineal nerve is stimulated. Those probe stimulations are referred to as:
1) The “OAB probe stimulation”-stimulation parameters directed toward managing overactive bladder syndrome, and
2) The “SUI probe stimulation”-stimulation parameters directed toward managing stress urinary incontinence.
3) The “FI probe stimulation”-stimulation parameters directed toward managing fecal incontinence.
4) The “POP stimulation”-stimulation parameters directed toward managing pelvic organ prolapse.

Specifically, the OAB probe stimulation and/or treatment stimulation may have a set of one or more of the following parameter specifications for humans:
an amplitude of between about 0.4 milliamps and 1 milliamp,
each pulse having a pulse duration of between about 200 microseconds and 400 microseconds (when the OAB probe stimulation is a periodic signal),
a frequency of between about 5 Hertz and 20 Hertz (when the OAB probe stimulation is a periodic signal), and
duty cycle of between about 5% and 100% (e.g., more specifically between about 8% and 12%).
a stimulation pulse of about 10-20 seconds followed by about 2.5 minutes off, repeated at least 3-4 times within a single session
a duration for transmitting the OAB probe stimulation of no less than 10 minutes and for no longer than 30 minutes in a single session.

These values can be scaled appropriately for different mammals. In addition, these values can be adjusted as a function of the number of treatments per day or week, the individual, and/or the disease severity.

The SUI probe stimulation and/or treatment stimulation is intended to introduce interference in the nerve signals that report bladder stretching to the brain. One or more stimulation pulses are delivered to the target nerve to interrupt the existing nerve signals and return the nerve to a relaxed state. The stimulation parameters may have a set of one or more of the following specifications for humans:
an amplitude of between about 0.5 milliamps and 2 milliamps,
each pulse having a pulse duration of between about 200 microseconds and 400 microseconds (e.g., when the OAB probe stimulation is a periodic signal),
a frequency of between about 40 Hertz and 100 Hertz (when the OAB probe stimulation is a periodic signal), and
duty cycle of between about 5% and 100% (e.g., more specifically between about 8% and 12%).
a stimulation pulse train of about 10-20 seconds followed by about 2.5 minutes off, repeated at least 3-4 times within a single session
a duration for transmitting the SUI probe stimulation of no less than 30 seconds and for no longer than 120 seconds in a single session.

As another example, the FI probe stimulation and/or treatment stimulation may have a set of one or more of the following specifications for humans:
an amplitude of between about 0.4 milliamps and 2 milliamps,
each pulse having a pulse duration of between about 200 microseconds and 400 microseconds (when the FI probe stimulation is a periodic signal),
a frequency of between about 2 Hz and about 80 Hertz (when the FI probe stimulation is a periodic signal), and
a duration for transmitting the FI probe stimulation of no less than 10 minutes and for no longer than 30 minutes in a single session.

These values can be scaled appropriately for different mammals. In addition, these values can be adjusted as a function of the number of treatments per day or week, the individual, and/or the disease severity.

POP stimulation affects nerves and muscles in the pelvic floor associated with support of pelvic organs. The POP probe stimulation and/or treatment stimulation may have a set of one or more of the following specifications for humans:
an amplitude of between about 0.4 milliamps and 4 milliamp,
each pulse having a pulse duration of between about 200 microseconds and 400 microseconds (e.g., when the POP probe stimulation is a periodic signal),
a frequency of between about 40 Hertz and 100 Hertz (e.g., when the POP probe stimulation is a periodic signal), and
a duration for transmitting the POP probe stimulation of no less than 7 seconds and for no longer than 30 seconds in a single pulse train, and between 1-4 pulse trains per session, and between 1-4 sessions per day of treatment.

As with the specifications for the OAB probe stimulation, these values can be scaled appropriately for different mammals. In addition, these values can be adjusted as a function of the number of treatments per day or week, the individual, and/or the disease severity.

Generically, a probe stimulation for neuromodulation of the pelvic floor may have a frequency of about 2 Hz to about 100 Hz. A nerve block signal may use higher frequencies, such as about 1 kHz to about 40 kHz. However, some embodiments may use low frequencies (e.g., <10 Hz for nerve blocking).

Advantageously, both afferent and efferent probe stimulations may be transmitted on the same nerve using the same neuromodulation device 100.

TABLE 1
TREATMENT PARAMETERS FOR FECAL INCONTINENCE
(FI) AND OAB IN HUMANS

| | Target | | | | Pulse | Duty | Treatment |
|---|---|---|---|---|---|---|---|
| | Nerve | Frequency | | Amplitude | Duration | Cycle | Duration |
| FI (afferent | Inferior | 2 | Hz - | Sub- | 200 | (a) 15 min | About 10 |
| probe | Rectal | 20 | Hz | threshold. | microsec. - | continuous | min. - |
| stimulation) | Nerve | | | Bi-Phasic | 300 | to | about 30 |
| and/or OAB | | | | pulse. | microsec. | (b) 15 sec | min. |
| (afferent | | | | 0.4 mA to | | ON, | 1-3 × a |
| probe | | | | 1.0 mA. | | 2. 5 min | day. |
| stimulation | | | | | | OFF, | |
| | | | | | | repeating 4 | |
| | | | | | | times | |
| FI | Inferior | 50 | Hz - | Threshold | 250 | 15 sec ON, | About 10 |
| (efferent | Rectal | 80 | Hz | Bi-Phasic | microsec. - | 2. 5 min | min. - |

| | | | | | |
|---|---|---|---|---|---|
| probe | Nerve | pulse. 0.5 | 400 | OFF, | about 13 |
| stimulation) | | mA to 2.0 | microsec. | repeating 4 | min. |
| | | mA | | times. | 1-3 × a |
| | | | | | day. |

Experimentation by the inventors has shown that efferent stimulation of the inferior rectal nerve using the above referenced stimulation parameters may close the external, and secondary rectal sphincter.

Details of various probe stimulations (also referred to as test signals) that may be used are described in co-pending U.S. patent application Ser. No. 17/985,843, which is incorporated herein by reference in its entirety.

At step 208, a physiological response to stimulation (e.g. probe stimulation, therapeutic stimulation, etc.) is detected and processed by the controller 400. In various embodiments, the controller 400 may receive multiple signals from one or more sensors. In some embodiments, the controller 400 receives data from one or more sensors 192 in combination. Sensor 192 information is utilized by a nerve stimulation system controller 400 to one or more of detect conditions suitable for nerve stimulation (i.e. stimulation triggering), quantify the effect of one or more nerve stimulations (e.g. detection and progress monitoring), and serve as the basis for determining the nerve stimulation parameters for one or more subsequent stimulations (e.g. feedback).

In some embodiments of a nerve stimulation system, an EMG sensor 192 is configured to detect one or more of an EMG signal from a target muscle innervated by the stimulator or a reflex muscle that has a reflexive EMG signal as a response to stimulation of the target muscle. In various embodiments, depending on the condition being treated, the EMG signal may be obtained from one or more muscles utilizing one or more sensors.

Exemplary muscles measured by EMG include but are not limited to the following:
OAB: detrusor muscle (e.g., to record bladder overactivity in OAB treatment).
FI: In the rectum: external and internal anal sphincter muscles within the rectum and/or the puborectalis muscle in the pelvic floor to treat FI.
SUI: external and internal urethral sphincter muscles in the urethra and/or the pubococygeous muscle in the pelvic floor to treat SUI.
Pelvic organ prolapse: coccygeus muscle, iliococcygeus muscle, pubococcygeus muscle, and/or puborectalis muscle to treat POP.
Gluteus maximus, quadricepts, soleus, gastric nemius, hamstrings for ambulation assistance.

Thus, as described above, in various embodiments, the sensors 192 may include, among other things, an EMG sensor, an ENG sensor, a pressure sensor/strain sensor, patient 102 movement sensor, relative movement sensor, and/or patient 102 feedback (e.g., through a user interface of dial/knob). The controller 400 receives data from one or more sensors 192 and uses the sensed data to modulate a therapeutic dosage. In some embodiments, the controller 400 receives data from one on more sensors 192 in combination.

The process then proceeds to step 210, where the treatment stimulation parameters are adjusted by the controller 400 as a function of the sensed physiological response in step 208. This may include a change in frequency, amplitude, or duration of stimulation in a given therapy session. For certain patients, the frequency should not change. For other patients, the frequency is adjusted to be lower. For example, if a sensed physiological signal indicates a Grade 4 prolapse, the patient's bladder is completely out (e.g., the patient 102 has to push it inside). Illustrative embodiments of treatment of weakened muscle (e.g. POP and SUI) thus start or adjust stimulation at a very low frequency to strengthen the muscle. This may also involve adjustments in patient 102 positioning, bladder status, or straining (e.g., patient 102 may adjust from sitting down and stimulation to sitting up and then stimulating).

After receiving the data, illustrative embodiments preferably provide a closed-loop stimulation and return to step 206. Preferably, adjustment occurs such that any pain or sensation is minimized in subsequent stimulations, but stimulation is sufficient to provide improved therapeutic outcomes. This can be considered to create a therapeutic window, such that the stimulation is high enough to have an effect but low enough to minimize side effects. In other words, the patient 102 feedback provides the controller 400 with information to titrate stimulation parameters. The controller 400 may then adjust stimulation based on the received signals and updated parameters. In some embodiments, this advantageously has the benefit of extending battery life of an implanted device 100 by not delivering more stimulation than is required.

Thus, various embodiments provide the following repeating loop until the desired outcome is achieved:
Initialization: The neuromodulation device 100 is activated (e.g., after it is implanted), and sends one or more probe stimulations to the patient 102 via the target nerve.
Monitoring: The sensor(s) continuously monitor the physiological response signals (e.g., signals discussed above) and send this data to the processor/controller. The processor uses algorithms to analyze the frequency, amplitude, and pattern of these signals.
   Various embodiments use inputs from multiple sensors. These sensor 192 inputs are quantified and utilized to determine the timing and parameters for the next stimulus. In one embodiment, individual sensor 192 data have weighting factors applied to them, and are combined in a quantitative manner to determine therapy. The weighting factor given to sensor 192 inputs may be experimentally determined on a patient 102 within the clinic or in the field. This could be done, for example, on a population basis and/or it may be possible to “calibrate” the feedback loop for each individual patient 102 at the time of surgical implantation where the surgeon can direct the stimulation and observe or measure the output and what the sensors 192 measure.
Detection and Response: When the processor detects an improvement in the function of the muscle, it triggers the stimulator to increase the intensity or change the pattern of stimulation. In some embodiments, this is done automatically without patient 102 intervention. When the processor detects degradation in the function of the muscle, it triggers the stimulator to cease stimulation, decrease the intensity of stimulation, or change the pattern of stimulation.
Adjustment: As the stimulator adjusts its output, the sensors 192 monitor the resultant changes in the (e.g., motor) signals. If muscular or nerve function improves, the stimulation adjustments may be maintained or fine-tuned. If muscular dysfunction persists or worsens, further adjustments are made.
Continuous Adaptation: In some embodiments, the system continuously adapts its responses based on real-time motor signal data, aiming to maintain optimal restorative motor function with minimal side effects. In some embodiments, this adaptation occurs on a periodic basis (e.g. daily), where the system analyzes the performance from the prior time period (e.g. 48 hours) and adjusts stimulation parameters accordingly.

Example of the process: sensors 192 in the pelvic nerve monitor the activity of the bladder. Frequency of the action potentials detected from pressure sensors 192 in the urothelium indicate maximal pressure and voiding frequency. OAB may be inferred by a low bladder pressure prior to voiding over one or more urinations and/or frequent intermicturition timing. In one embodiment, if OAB is detected (e.g., inferred by the nerve stimulation system), the system starts OAB treatment on the afferent branches of one or more target nerves, such as the perineal nerve. EMG signals recorded from sensors 192 monitoring any of the pelvic floor muscles including the cocyggeous muscle can be used to determine the status (normal vs injured), function (weak or strong contraction) and evoked response to direct electrical stimulation (normal or abnormal nerve fiber recruitment). These signals can then be used to titrate the amount of stimulation energy that is delivered to the muscle (i.e., less to a weak muscle, and more to one that has been recovered). As an example, the stimulation current may be gradually increased from 0.25 mA if the EMG signal is 50% of that of normal values, and increased 0.10 mA with every 10% of increase in the EMG signal recorded until the 100% stimulation amplitude and frequency is achieved. In this example, EMG negative signals are not expected since the denervated muscles do not degenerate completely, mostly hypotrophy to 50% of normal. Neural signals, if negative (failed to evoke a nerve action potential after stimulation), can indicate nerve injury (illustrative embodiments may then discontinue the treatment and refer to a surgical revision) or a failure of the device 100 (refer to the technical team to evaluate the mechanical and electrical functioning of the device).

The process then comes to an end.

It should be apparent to one skilled in the art that the closed loop stimulation of various embodiments advantageously evokes a response, evaluates the response, and adjusts therapy based on the response of the patient. This is in contrast to other closed-loop methods which continuously scan for abnormal motor signals (e.g., suggestive of Parkinson symptoms) and trigger a stimulator to reduce symptoms. Illustrative embodiments may also provide such a closed-loop stimulation. Preferably, however, illustrative embodiments provide a probe stimulation configured to elicit feedback/a physiological response. The response is detected by one or more sensors, and used to adjust the course of stimulation going forward over time to modify therapeutic stimulations for restorative effect to the nerve, nerve junction, and/or affected tissue (e.g. muscle, organs). Thus, while a reduction in symptoms may occur, this is not a result of detecting the symptom, but instead detecting the body's response to the probe signal. Furthermore, illustrative embodiments advantageously provide highly targeted nerve stimulation, as is discussed herein.

In various embodiments, the neuromodulation system includes closed-loop functionality that dynamically adapts stimulation parameters based on diagnosed physiological states of a target muscle or nerve region. Unlike systems that trigger stimulation based solely on the detection of a specific event or biosignal crossing a preset threshold, illustrative embodiments leverage diagnostic feedback to assess muscle, neuromuscular junction, and/or nerve condition over time and to titrate therapy accordingly.

Specifically, biosignal data—such as EMG, ENG, or strain measurements—may be analyzed to determine whether a muscle is fatigued, weak, overstimulated, underactive, or strengthening. Based on this diagnosis, the controller 400 adjusts one or more stimulation parameters (e.g., amplitude, frequency, pulse duration, duty cycle) to optimize therapy delivery and/or avoid fatigue or unnecessary stimulation rendering the therapy less effective. This adaptive titration may occur between therapy sessions or in real time, allowing the system to progressively adjust stimulation (e.g., adjust stimulation as the muscle, nerve, and/or neuromuscular junction heal or scale it back if fatigue is detected).

In various embodiments, EMG signals can be used to determine whether a muscle is fatigued, weak, overstimulated, underactive, or strengthening. EMG can also be used to determine how well the stimulation signal is being transferred to the muscle as an indicator of nerve or neuromuscular junction health.

In various embodiments, the system includes at least one EMG sensor positioned to monitor electrical activity in a muscle innervated by a target nerve. EMG signals are acquired before, during, and optionally after stimulation, and may be used to determine the physiological state of the muscle through signal processing and over time via trend analysis.

The EMG signal may be amplified, filtered (e.g., 50-500 Hz bandpass), and rectified. A linear envelope of the signal may be generated, representing the overall intensity of muscle activation over time. The controller 400 may assess one or more features of the EMG signal to determine the muscle's condition:
Muscle fatigue may be determined by a progressive decrease in EMG amplitude across repeated contractions or stimulation trains within a therapy treatment. For example, if the EMG envelope decreases by more than 20% during a fixed stimulation regimen, the system may infer that the muscle is entering a fatigued state and adjust therapy accordingly.
Muscle weakness may be determined when EMG amplitude remains consistently low during attempted voluntary or evoked contraction, particularly if the signal fails to increase after multiple sessions or increases only minimally in response to increased stimulation amplitude.
Overstimulation may be determined when abnormally high EMG amplitudes are observed over extended stimulation durations or when signal quality degrades despite increasing stimulus, suggesting a loss of functional efficiency or neuromuscular overexertion.
Underactivity may be determined in cases where EMG activity is below a baseline threshold or absent altogether in response to stimulation known to be suprathreshold, indicating poor recruitment, signal conduction failure, or non-participation of the target muscle.
Strengthening may be determined from progressively increasing EMG amplitude in response to the same or decreasing stimulation intensity, or from shorter time-to-peak values (i.e., the time from stimulation onset to peak EMG response), indicating improved nerve conduction or muscle responsiveness.

By evaluating these EMG-derived features either in real time or across therapy sessions, the controller 400 can execute a closed-loop titration protocol, adjusting stimulation parameters—such as amplitude, frequency, pulse width, train duration, or number of trains—to optimize therapy delivery and prevent overstimulation or fatigue. Additionally, OAB signals are intended to be sub-threshold. As the nerve heals or undergoes regeneration, the sub-threshold amplitude signal, but eventually become supra-threshold at the same amplitude. At this point the EMG signal will provide feedback to reduce the stimulation amplitude for the sub-threshold portion of the therapy signal.

In various embodiments, ENG sensors 192 can be used to determine whether a muscle is fatigued, weak, overstimulated, underactive, or strengthening. In some embodiments, the system utilizes one or more ENG sensors to monitor neural activity within or adjacent to a target nerve. ENG signals may be used to evaluate nerve responsiveness, conduction velocity, and recruitment efficiency in response to either natural voluntary commands or externally delivered neuromodulation therapy.

ENG signals are typically recorded via cuff electrodes or microelectrodes positioned along the nerve or its branches. After acquisition, the signal is amplified, filtered, and digitized to extract features such as:
Signal amplitude, representing compound action potential magnitude;
Latency, representing time between stimulation onset and neural response;
Conduction velocity, derived from propagation time between spatially spaced electrodes.

These signal characteristics may be analyzed over time to infer various physiological states:
Fatigue or desensitization may be determined by an increase in response latency or a decrease in compound action potential amplitude after prolonged stimulation.
Weakness or poor nerve recruitment may be determined by low-amplitude or absent ENG signals, even when stimulation intensity is above threshold.
Overstimulation may be determined from erratic or non-monotonic increases in signal amplitude despite increased stimulation intensity, or the presence of irregular firing patterns.
Strengthening or recovery may be determined by a reduction in response latency, increase in signal amplitude, or greater consistency of neural firing over time in response to fixed stimulation intensity.

These analyses allow the controller 400 to tailor therapy parameters—such as intensity, frequency, or targeting location—based on the evolving physiological characteristics of the nerve.

In some embodiments, the system includes strain sensors configured to monitor biomechanical deformation or tissue displacement associated with muscle contraction, pelvic floor elevation, or other organ-supporting movement. These sensors may be affixed across soft tissue structures or implanted along fascial planes, muscle bellies, or ligamentous supports.

Strain signals may be used to detect:
Muscle contraction strength, based on peak strain during evoked contraction;
Tissue recoil or relaxation dynamics, based on the return profile after contraction;
Baseline tone, through low-level continuous tension in resting states.

The following diagnostic interpretations may be supported:
Fatigue may be determined by a reduction in peak strain or slower recovery time after repeated contractions.
Weakness or underactivity may be determined by minimal strain displacement in response to known suprathreshold stimulation.
Strengthening or improved recruitment may be determined from increased strain amplitude or faster strain rate over time.
Asymmetrical movement across paired strain sensors may determine imbalanced muscle activation, relevant for bilateral treatment strategies.

By integrating strain data with EMG and/or ENG, the system can execute multi-modal sensor fusion, improving diagnostic accuracy and refining the closed-loop control strategy for individualized therapy adjustment.

Various embodiments utilize a combination of two or more biosensors to enhance the accuracy, resolution, and context of physiological state assessments. For example, EMG may be used to monitor the electrical activity of muscles during evoked contractions, ENG may be used to assess neural signal conduction along the target nerve, and strain sensors may be used to detect the mechanical deformation or displacement of tissue or pelvic floor structures during muscle activation. By integrating multiple types of signals—e.g., EMG and ENG, or ENG and strain—the controller 400 can implement sensor fusion algorithms to corroborate findings, resolve ambiguities, or identify state-dependent patterns that would not be evident from a single signal source alone. For instance, a reduction in EMG amplitude accompanied by delayed ENG latency and reduced strain displacement may provide high-confidence identification of muscle fatigue or weakness. Conversely, increased strain amplitude alongside improved ENG responsiveness and stable EMG thresholds may indicate functional strengthening. This multi-sensor framework enables more robust and precise diagnosis of fatigue, underactivity, overstimulation, or strengthening, and supports highly individualized therapy modulation across sessions or in real time.

### Example 1: Closed-Loop Therapy for Pelvic Organ Prolapse (POP)

In various embodiments, a closed-loop neuromodulation system is configured to treat pelvic organ prolapse (POP) by targeting nerves that innervate pelvic floor muscles, such as the pubococcygeus and puborectalis. The neuromodulation device 100 may be coupled to one or more distal branches of the perineal nerve and/or branches of the levator ani nerve.

In some embodiments, the system includes one or more EMG sensors 192 coupled to the target muscle, such as the pubococcygeus or puborectalis, to monitor muscular response to electrical stimulation. Optionally, one or more strain sensors 192 may be deployed to detect tension in pelvic ligaments to evaluate the mechanical support being provided by the muscle group. The sensor may include a thin metallic membrane 79.

During therapy, the system continuously samples EMG signal amplitude from the target muscle. If the EMG signal amplitude falls below a defined threshold (e.g., less than 50 μV during voluntary contraction), the neuromodulation device 100 delivers a stimulation pulse at 0.25 milliamps, 50 Hz, and 300 microsecond pulse duration. If the EMG signal is between 50-75 μV, stimulation parameters are adjusted to 0.35 milliamps, 60 Hz, and 300 microsecond pulse duration. If the signal exceeds 75 μV, indicating strong muscle activation, the system reduces stimulation to 0.2 milliamps at 40 Hz and 250 microsecond pulse duration.

In certain embodiments, fatigue is detected when EMG amplitude decreases by 20% or more within a therapy session. In such cases, stimulation is paused for a period of three minutes and may be resumed with adjusted parameters. Stimulation cycles may be timed relative to bladder filling states, such that stimulation coincides with higher intra-abdominal pressures to simulate functional load on the pelvic floor muscles.

### Example 2: Closed-Loop Therapy for Overactive Bladder (OAB)

In another embodiment, a closed-loop neuromodulation system is used to manage overactive bladder (OAB) by targeting one or more distal branches of the pudendal nerve. The system includes at least one bladder pressure sensor, strain sensor, or EMG sensor configured to detect signs of involuntary bladder contractions. In some embodiments, a pressure sensor is implanted in the bladder or urethra to monitor intravesical pressure; in others, a strain sensor monitors changes in bladder wall tension, or a bladder EMG sensor monitors detrusor muscle activation.

The controller 400 is configured to receive input from one or more of these sensors and to evaluate whether the bladder exhibits signs of overactivity. In particular, the system may detect frequent, transient increases in bladder pressure (e.g., three or more spikes above 20 cmH₂O within a 10-minute window) or repetitive EMG bursts from the bladder wall that are inconsistent with normal voluntary voiding behavior. These signals are interpreted as evidence of bladder overactivity.

When such overactivity is detected—e.g., elevated pressure events occurring without an indication of voluntary voiding intent—the system initiates stimulation through a distal pudendal nerve interface at 10 Hz, 0.8 milliamps, and 250 microsecond pulse duration, with a duty cycle of 15 seconds ON and 2.5 minutes OFF. In this context, absence of voluntary voiding intent may be inferred from a lack of coordinated pelvic floor EMG activation, absence of input from a user interface (e.g., button or mobile device), or deviation from a known voiding schedule.

When bladder overactivity persists despite initial stimulation—e.g., repeated bursts continue to occur, or bladder EMG signal quality degrades—the stimulation frequency may be increased to 12 Hz for three additional cycles before reassessment. If the patient provides input via a user interface (e.g., dial, button, or mobile app) indicating discomfort or excessive stimulation, the controller 400 may automatically reduce amplitude by 20% in subsequent therapy cycles.

Pressure, EMG, and/or strain signals may be sampled at one-second intervals or according to a programmable sampling schedule. The therapy regimen is continuously or sessionally adapted based on individual patient profiles, with the goal of suppressing premature detrusor overactivity and improving voluntary bladder control through targeted, personalized stimulation.

### Example 3: Closed-Loop Therapy for Stress Urinary Incontinence (SUI)

In certain embodiments, a closed-loop neuromodulation system is configured for the treatment of stress urinary incontinence (SUI). This system utilizes a miniaturized control circuit capable of both stimulating and sensing, such that electromyography (EMG) signals from pelvic floor muscles are continuously monitored during therapy.

The system may be operably connected to the pubococcygeus, coccygeus, puborectalis, or iliococcygeus muscles via implanted electrodes, which are in turn innervated by branches of the pudendal nerve, levator ani nerve, or other pelvic-specific nerves. These muscles contribute both to pelvic floor support and sphincteric function and may be differentially targeted depending on patient 102 anatomy or condition severity.

During therapy, electrical stimulation is delivered via a distal neurostimulator coupled to a nerve clip or a flexible perineural interface. Simultaneously, the system records EMG signals from one or more of the target muscles. The EMG signal is rectified and bandpass filtered (50-500 Hz), amplified, digitized, and processed to derive a linear envelope representative of muscle contraction strength.

The system is configured to compare real-time EMG envelopes to a baseline (established during initial calibration or voluntary contraction). If the EMG envelope decreases by more than 20% during evoked stimulation—indicating potential muscle fatigue or overstimulation—the system activates a closed-loop algorithm that modifies the stimulation protocol.

In response to fatigue detection, the closed-loop controller 400 may execute one or more of the following safety adjustments:
Reduce stimulation amplitude to 25-50% of the prior threshold level
Decrease the number of stimulation trains (e.g., from 4 to 3 or 2 per session)
Shorten the total stimulation duration (e.g., reduce from 10 minutes to 6 minutes)
Reduce stimulation frequency per day or per week

In other embodiments, a neural signal (e.g., electroneurogram, or ENG) may be used as the feedback signal instead of or in conjunction with the EMG. This enables the closed-loop algorithm to account for changes in neural firing thresholds, conduction velocity, or activation patterns.

The system therefore ensures adaptive therapy that avoids overstimulation, minimizes the risk of nerve damage or muscle fatigue, and enhances long-term neuromuscular rehabilitation by providing dynamic, on-demand control based on real-time physiological feedback.

### Example 4: Closed-Loop Therapy for Pelvic Organ Prolapse (POP) with Satellite

### Neuromodulation Devices

In another embodiment, a closed-loop neuromodulation system is configured for the treatment of pelvic organ prolapse (POP) using a distributed or “satellite” device 100 architecture. This architecture includes a primary neuromodulation module that houses a controller 400, power source, and communication circuitry, and two or more satellite stimulators, each operatively coupled to distal nerve branches via miniature neuroclips or flexible interfaces.

The system is designed to selectively target pelvic floor muscles (PFMs) that contribute to both structural support and sphincteric function. Relevant muscles include the pubococcygeus, puborectalis, iliococcygeus, and coccygeus, each innervated by branches of the perineal nerve, levator ani nerve, or related pelvic nerves. Satellite stimulators are implanted at or near these nerve branches, such that each muscle group can be independently stimulated and monitored.

At least one of the satellite modules—or, in some cases, the central controller 400—is further configured to receive biosignals, including electromyography (EMG) or electroneurogram (ENG) signals, from one or more of the target muscles. In some embodiments, these biosignals are acquired using multi-contact electrodes fabricated from graphene or other conformable conductive materials, enabling precise recording even in anatomically constrained or neurovascular regions.

The EMG signal is rectified, filtered within a 50-500 Hz band, amplified, digitized, and processed to derive a linear envelope indicative of muscle activation. The central controller 400 or designated satellite node evaluates these envelopes in real-time or near-real-time. If a given signal demonstrates a decrease in amplitude—such as a 30% drop relative to baseline—it is interpreted as a marker of fatigue, nerve adaptation, or insufficient muscle response.

In response, the central controller 400 issues updated control parameters to one or more satellite stimulators to adjust therapy as follows:
Reduce stimulation amplitude at the affected satellite (e.g., by 10%, 30%, or 60%).
Reduce the number of stimulation trains delivered by that satellite (e.g., from four to two per therapy session)
Temporarily suspend stimulation to the affected site while continuing therapy at unaffected sites
Alter pulse frequency or duty cycle to allow for localized recovery while preserving total session benefit

The satellite architecture enables independent and asynchronous control of each muscle region. For example, the pubococcygeus-targeting stimulator may enter a low-power recovery mode if fatigue is detected, while the puborectalis-targeting node continues its therapy regimen uninterrupted. In some embodiments, EMG signals are cross-referenced between sites to detect compensatory activation or synergistic muscular behavior, which can further inform stimulation logic.

Additionally, the system may respond to functional triggers such as increased intra-abdominal pressure, posture changes, or patient-initiated therapy via user input. These triggers may activate a full-cycle stimulation protocol or prompt selective activation of satellite nodes based on previously identified weakness or fatigue zones.

The use of satellite devices in this closed-loop architecture provides multiple advantages, including miniaturization of implantable components, reduced surgical complexity, localized and patient-specific targeting, and optimized energy delivery by offloading power generation and signal processing to the central module. This distributed system architecture is particularly well-suited for complex, multi-muscle disorders such as POP, where coordination of multiple stimulation targets is critical for effective treatment.

### Example 5: Closed-Loop Therapy for Pelvic Organ Prolapse with Longitudinal Adaptation

In one embodiment, a neuromodulation system is used to treat pelvic organ prolapse (POP) by targeting two pelvic floor muscles: the pubococcygeus and puborectalis. A principal neuromodulation device 100 is implanted on a large pelvic nerve trunk (e.g., sacral plexus or pudendal nerve) and wirelessly controls two satellite neurostimulation devices. One satellite device 100 interfaces with a distal branch of the perineal nerve (targeting the pubococcygeus), while the second interfaces with a distal branch of the levator ani nerve (targeting the puborectalis). Each device 100 includes a bipolar cuff electrode and receives power and control signals from the principal device.

In addition, an implantable EMG sensor is embedded in the pubococcygeus muscle and communicates wirelessly with the principal device. A second sensor, a strain sensor, is anchored across the pelvic floor and measures displacement during contraction events.

During the initial calibration session, probe stimulations are delivered with the following default settings: 0.5 mA amplitude, 250 us pulse width, and 20 Hz frequency, in a burst train of 5 seconds ON/10 seconds OFF, repeated 4 times. The EMG sensor records response latency and amplitude. Baseline response is defined as a peak EMG amplitude of 85 μV occurring 140 ms after stimulation onset, with a strain response of 0.8 mm displacement.

The controller 400 uses this data to set initial therapy parameters for the first week of treatment:
Amplitude: 0.6 mA
Frequency: 25 Hz
Pulse width: 275 μs
Train schedule: 6 seconds ON/8 seconds OFF, 4 repetitions per session, once daily

After seven days, the EMG sensor data shows a consistent reduction in response latency to 110 ms and an increase in peak EMG amplitude to 105 μV. Strain sensor readings increase to 1.2 mm, indicating stronger and more timely contractions. Based on these improvements, the system automatically updates the therapy regimen for the second week:
Amplitude: 0.8 mA
Frequency: 30 Hz
Train schedule: 8 seconds ON/6 seconds OFF, 5 repetitions per session, once daily

In the third week, the EMG amplitude begins to plateau at 108 μV, but strain response continues to increase, suggesting that some muscles may be compensating more than others. The system responds by asymmetrically adjusting the therapy:
Pubococcygeus stimulator (via perineal nerve): amplitude held at 0.8 mA, but frequency reduced to 25 Hz
Puborectalis stimulator (via levator ani nerve): amplitude increased to 1.0 mA, frequency increased to 35 Hz, with an additional 2 train cycles per session
   Over a six-week period, the therapy adapts weekly, using cumulative EMG and strain data to determine whether to escalate, maintain, or taper stimulation. At week six, the patient 102 achieves full restoration of functional lift as measured by EMG amplitude >120 μV, latency <95 ms, and strain displacement of 1.5 mm.

In some embodiments, the principal controller 400 transmits therapy summaries to an external mobile device 100 used by the clinician, allowing for long-term therapy review, override of automatic parameters, or patient-specific goal-setting.

It should be apparent that over the course of therapy, in various embodiments the system progressively adapts stimulation delivery based on physiological indicators of muscle function, neuromuscular engagement, or therapy effectiveness. These adaptations may include adjustments to stimulation amplitude, frequency, pulse width, train duration, train count, inter-train rest intervals, or session frequency, among other things. The changes may be made independently for different nerve targets or coordinated across multiple muscles to optimize therapy balance. For example, if muscle activation improves more rapidly in one region than another, the system may shift stimulation intensity to focus on the lagging muscle group. Conversely, if fatigue or overstimulation is detected in one target, therapy intensity may be reduced locally while maintaining stimulation elsewhere. This dynamic modulation supports progressive muscle strengthening, functional recovery, and reduced risk of overstimulation or fatigue, enabling a closed-loop system that evolves in response to changing patient 102 needs across therapy sessions.

The foregoing examples are provided for the purposes of illustration and are not intended to be limiting. In various embodiments, the described closed-loop neuromodulation systems and methods may be adapted to treat a range of pelvic conditions, including but not limited to pelvic organ prolapse (POP), stress urinary incontinence (SUI), overactive bladder (OAB), and fecal incontinence (FI). The specific muscles and nerves targeted may vary depending on patient 102 anatomy, clinical indication, or therapeutic strategy.

For example, in different embodiments, stimulation and/or sensing may be applied to one or more of the following pelvic nerves and their distal branches:
Perineal nerve (e.g., for POP and SUI, targeting the pubococcygeus and puborectalis muscles)
Pudendal nerve and its branches (for SUI, OAB, and FI)
Inferior rectal nerve (for FI)
Levator ani nerve (for POP and FI)
Coccygeal nerve (for pelvic floor support in POP)
Sacral nerve roots (S2-S4) (especially for bladder and bowel control in OAB and FI)

Stimulation parameters used across embodiments may be selected or adjusted based on patient 102 response, tissue type, electrode configuration, and therapy goals. Unless otherwise stated, typical stimulation parameters include:
Amplitude: approximately 0.1 mA to 5.0 mA
Pulse width: approximately 50 microseconds to 600 microseconds
Frequency: approximately 2 Hz to 100 Hz
Train duration: approximately 1 second to 30 seconds
Inter-train interval: approximately 1 second to 60 seconds
Total treatment duration per session: approximately 30 seconds to 30 minutes

One or more of these parameters may be modified dynamically based on real-time feedback from one or more physiological sensors, including electromyography (EMG), electroneurography (ENG), bladder pressure sensors, strain sensors, accelerometers 77, and electrochemical sensors. Modifications may be implemented autonomously by a controller 400 located within an implanted device, or externally by a patient-facing or clinician-facing interface.

Various embodiments provided advantages over closed-loop neuromodulation systems that operate in a reactive, event-driven manner—e.g., where stimulation is delivered immediately in response to the detection of a specific biosignal, such as a spike in nerve activity or EMG amplitude. Such systems typically follow a logic model of: detect event→trigger stimulation.

Desirably, various embodiments described herein are not dependent on real-time event detection alone. Instead, the system collects biosignal data over time and applies a diagnostic framework to assess underlying physiological status. For example, the system may monitor a decline in EMG signal amplitude across multiple contractions and interpret this as muscle fatigue, triggering a reduction in stimulation intensity or frequency. Alternatively, a gradually increasing EMG amplitude across sessions may indicate improved muscle strength, prompting a staged increase in therapy intensity. This condition-aware, titration-based approach enables a higher degree of personalization and therapy optimization, particularly for indications such as SUI, POP, OAB, or FI.

Therefore, the closed-loop methods disclosed herein represent a diagnostic and adaptive model rather than a purely reactive one. However, some embodiments may limit closed-loop feedback to event triggers rather than physiological state evaluation and longitudinal titration.

Some embodiments may provide a controller 400 that deliver neuromodulation therapy in response to the detection of acute events-such as imminent urinary leakage, patient movement, or a spike in EMG activity. However, preferably, illustrative embodiments do not merely detect a discrete biosignal threshold and trigger a reactive stimulation response. Instead, the controller 400 is configured to continuously or periodically analyze biosignals (e.g., EMG, ENG, strain) to diagnose the physiological state of one or more target muscles or nerves. This diagnosis may include determining whether the muscle is fatigued, weak, overstimulated, underactive, or strengthening, based on temporal trends, response thresholds, amplitude profiles, or latency metrics derived from the biosignals.

The system further includes logic to titrate therapy over time in accordance with these diagnosed states. For example, if a muscle is found to be fatigued (e.g., based on a >20% drop in EMG amplitude), the system may reduce stimulation amplitude or frequency until recovery is achieved. Conversely, if strengthening is detected (e.g., via improved time-to-peak EMG response or reduced stimulation threshold for evoked contraction), the system may progressively increase stimulation intensity to continue challenging the target musculature. This approach allows for longitudinal adaptation of therapy, providing a therapeutic effect that evolves with the patient's recovery or functional progress, rather than simply augmenting insufficient natural effort on a moment-by-moment basis.

Moreover, certain embodiments disclosed herein support coordinated, multi-node stimulation of different pelvic floor muscles using a distributed neuromodulation architecture. Each node may operate independently or under centralized control, and may use a variety of biosensors—including strain sensors and ENG detectors—not contemplated in prior art. Thus, the illustrative embodiments of the closed-loop system provide a diagnostically-driven, multi-modal, and condition-adaptive therapy platform, distinctly different from reactive systems that rely solely on event detection and reflexive stimulation.

The inventors determined that selective stimulation of certain nerves leads to improvements in symptoms of pelvic organic prolapse. Pelvic organ prolapse (POP) involves the displacement of pelvic organs due to the weakening or damage of the muscles and connective tissues in the pelvic floor.

POP involves one or more pelvic organs, such as the uterus, bladder, or rectum, descending or protruding into the vaginal space. The treatment pathway is similar to SUI at early stages. In postmenopausal women, hormone therapy improves the strength and elasticity of vaginal tissues. Solutions to POP have been limited. For example, a pessary, a device 100 inserted into the vagina to support the pelvic organs, can alleviate symptoms. The surgical implantation of mesh was previously used as a definitive treatment of POP but has now been restricted or banned in many countries due to the long-term complications. Female sexual disfunction is treated with hormone therapy (e.g. flibanserin (an antidepressant that can cause low blood pressure, sleepiness, nausea, and fatigue) or bremelanotide which requires an injection with nausea as a possible side effect). In a survey of 5,236 women 20 years post-partum, Gyhagen et al., 2015, found that the prevalence of pelvic floor disorders was 46.5%, and 14.8% had more than one disorder with a higher prevalence for vaginal delivery (17.1%) compared with cesarian delivery (8.4%). However, no simultaneous, coordinated, or integrated therapy for multiple disorders is currently available.

To that end, various embodiments treat POP by strengthening the pubococcygeus muscle. Strengthening the pubococcygeus muscle helps support the entire pelvic floor. The inventors stimulate one or more of the pelvic floor muscles: the Pubococcygeus, Puborectalis, Iliococcygeus, and the Coccygeus (Ischiococcygeus) muscle to improve POP. The pubococcygeous muscle provides the proximal sphincter function and the other muscles add to the support of the pelvic organs. In normal healthy patients, these muscles are engaged during sudden increments in intra-abdominal pressures. In some embodiments, a nerve stimulation system stimulates these muscles when sudden increments in intra-abdominal pressures are sensed.

Various embodiments of this nerve stimulation technology may begin treatment when the patient 102 has a mild/early-stage form of prolapse. Early-stage prolapse can be detected using EMG or profilometry.

The combination of 1 or more stimulators on the nerves that innervate the aforementioned muscles can help restore neuromuscular function, strengthen to prevent worsening, and/or restore pelvic organ support (i.e., for pelvic organ prolapse). Similar approaches can be applied to the stimulation of one or more organs, and muscles throughout the body to restore and/or augment function.

Illustrative embodiments start with softer treatment for POP in the most severe cases (e.g., to prevent ripping the muscle or further damage) relative to SUI.

Electrical stimulation parameters may also be tied to load on the muscle. The inventors determined that electrical stimulation of the muscle with load on the muscle can provide considerable therapeutic benefits. In the case of the urethral sphincter, for example, the bladder is filled several times a day, which creates pressure or load against the sphincter. For the pelvic floor, illustrative embodiments time the stimulation when the bladder is the fullest/to work with elevated load as well.

Illustrative embodiments prevent or repair pelvic organic prolapse by stimulating one or more muscles via innervating nerves. In particular, the pubococcygeus and puborectalis. Other targets include a coccygeal nerve and an ischeal nerve, nerves that have branches that extend to the muscles.

The inventors have determined that:
(1) the pelvic floor is dually innervated by 2 large nerves, the levator ani and the perineal nerve. The levator ani provides a lot of those branches from the posterior part, and the perineal nerve provides a lot of the other branches (which is part of the pudendal) from the anterior. Conceptually, the muscle may be thought of as a bowl. The back of the bowl is innervated by the levator ani, and the front is innervated by the perineal nerve.
(2) These muscles can be activated directly and individually, or they may be activated via a reflex activation. In other words, one muscle is activated, and others are activated by a reflex action.

Various embodiments may put a stimulator on one nerve, or put a stimulator on each of 2 nerves, 3 nerves, 4 nerves, or more. FIG. 1M schematically shows a plurality of nerve targets for treatment of POP, OAB, SUI, and/or

FIG. 1N schematically shows a chart of nerve targets for treatment of various conditions with the use of neuromodulation devices 100 in accordance with illustrative embodiments.

FIG. 2B shows innervation patterns of target nerves in accordance with illustrative embodiments of the invention. As shown, the pubococcygeus muscle is innervated by branches of the perineal and coccygeal plexus at different ends. While the pelvic nerve is a branch of the perineal plexus innervating the anterior Pcm, it also receives innervation of the pubococcygeus nerve, which branch from the levator ani. The percentage innervations are based on the two references listed in the slide.

In various embodiments, closed-loop stimulation may be used to treat POP. In this way, patient 102 progression in therapy may be automated. As described previously, a probe stimulation may evoke a neuromuscular response (e.g., detected EMG, ENG, etc.) to see if muscle function is improving with stimulation. Based on that improvement the parameters may be adjusted automatically. Some embodiments may use patient 102 feedback to determine improvement in function. The closed-loop process continuously senses and continuously stimulates and continuously refines itself.

As discussed above, various embodiments use sensing to calibrate treatment. The sensing does not need to be continuous. In some embodiments, sensing is utilized periodically (e.g. once per day, once per hour) or under certain conditions (e.g. patient 102 sleeping, resting, walking). Illustrative embodiments stimulate the muscle (e.g. probe or treatment stimulation), and detect (e.g. through sensors 192 or recording electrodes) how well the muscle responds to the stimulus (this also provides information on the condition of the muscle). In one embodiments, stimulations are performed continuously and used to evaluate whether the muscle is strengthening and/or growing. The controller 400 then decides whether to increase or decrease therapy depending on how well the muscle is performing.

For example, in some embodiments, a nerve stimulation controller 400 measures muscular response by measuring electromyographic (EMG) activity with a sensor 192 when the perineal or levator ani nerve is stimulated. The response gets faster (i.e., less time before peak EMG amplitude is reached) after a period of time receiving nerve stimulation therapy, indicating that the muscle is strengthening. As the nerve stimulation system detects faster response times, the system increases the level of nerve stimulation by altering one or more stimulation parameters, such as pulse amplitude, frequency, or train duration, thereby progressively increasing the muscular effort induced by stimulation in step with muscle strengthening.

In some treatments, the nerve is partially injured and it recovers over time. Illustrative embodiments sense how well the nerve is conducting, how well the muscle is contracting, and/or if other muscles are reflexively contracting (all in response to the stimulation). To make this determination, illustrative embodiments may detect EMG, ENG, nerve conduction velocity, sphincter closure pressures, and/or electrochemical sensing (e.g., by using graphene fiber electrodes). Examples of electrochemical sensing include detecting neurotransmitters such as dopamine, norepinephrine, and/or acetylcholine.

In various embodiments, the one or more neuromodulation devices may include a metalized (i.e. jacketed) electrically conductive fiber (e.g. graphene, carbon nanotubes (CNTs), silicone, metal-free fibers like polyaniline (PANI), or hybrid composites (MXenes and cellulose)) to create flexible and free-standing microelectrode arrays (e.g., with a thin platinum coating). These electrically conductive fibers offer unique mechanical and electrochemical properties, enhancing electrode performance compared to conventional materials. In one embodiment, the porous structure of graphene fibers provides an a large charge injection capacity, improving the ability to record and detect neuronal activity, while the thin platinum layer efficiently transfers collected electrons along the microelectrode. In some embodiments, graphene fibers are manufactured using liquid crystalline dispersions of graphene oxide.

Advantageously, electrodes comprising metallized electrically conductive fibers (e.g., graphene) can be used for cell-culture recordings, biochemical biosensing, and measuring various biomarkers like neurotransmitters and reactive oxygen species. Furthermore, electrically conductive fibers can be utilized for neuromodulation of somatic and autonomic ganglia, direct neuromodulation of organs, and as implantable conductive sutures for neural interfaces, providing adaptability and high electrode sensitivity.

Processes for forming using the graphene fiber electrodes are described in greater detail in U.S. patent application Ser. No. 16/691,309, which is incorporated herein by reference in its entirety. It should be understood that one or more of the electrodes of the one or more neuromodulation devices described in this application may include an electrically conductive fiber.

Additionally, the graphene fiber electrodes may also be used to provide a stimulation, as well as for recording/receiving of signals.

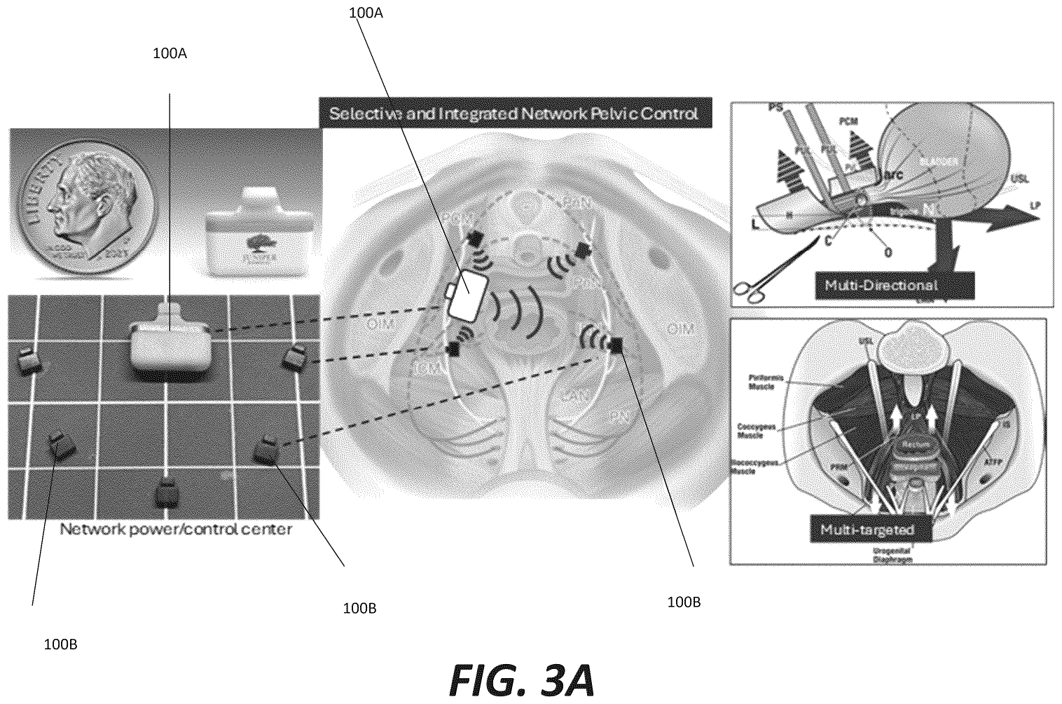

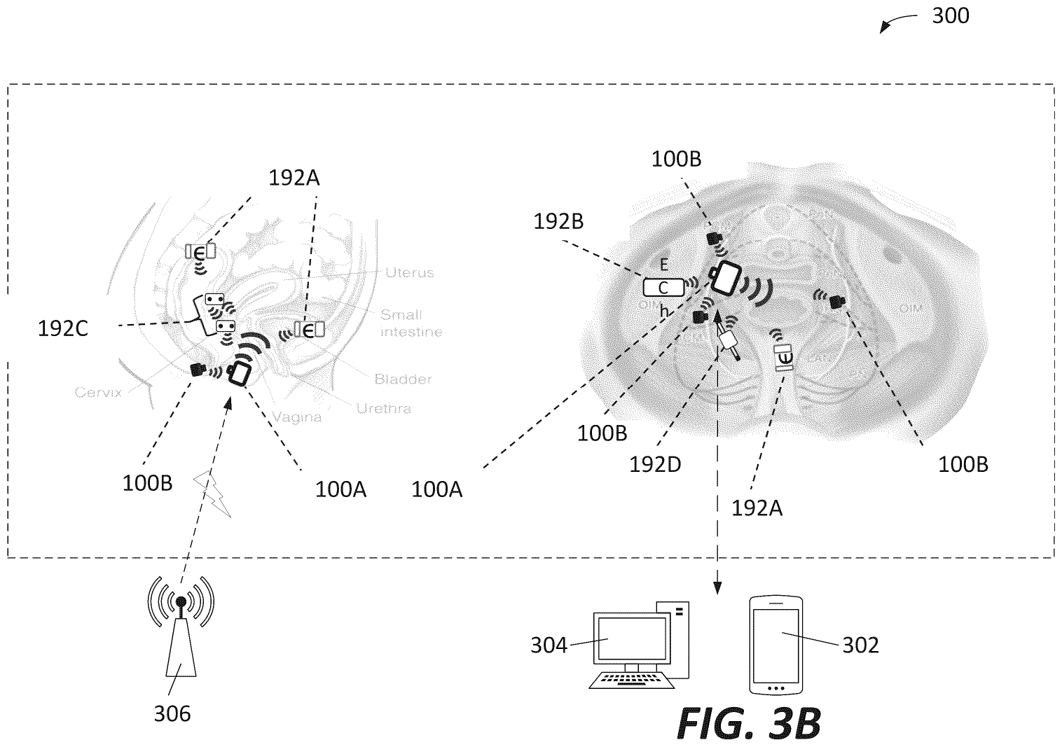

FIGS. 3A-3B schematically show a system 300 for neuromodulation in accordance with illustrative embodiments. FIG. 3B shows details of additional sensors 192 that may be separate from the neuromodulation device 100. The left side of FIG. 3B shows a sagittal plane device array example. The right side of FIG. 3B shows a pelvic floor device array example. Among other things, FIG. 3B shows:
a primary neuromodulation device 100A. The device 100A may be coupled to the nerve using the Slide-and-Lock method to anchor the electrode and/or sensor 192 to the nerve. This device 100A may be battery operated.
a plurality of satellite neuromodulation devices 100B. These may be miniature-devices. The device 100B may be coupled to the nerve using the Slide-and-Lock method to anchor 43 the electrode and/or sensor 192 to the nerve.
Strain sensors 192A on the bladder and rectum. A pressure membrane 73 connected to a semistretchable membrane that has two anchor 43 points as seen in the image indicates stretch. A strain sensor 192 can detect strain in ligaments, muscles, bladder wall, colon wall, or the rectum wall. The sensor 192 may be anchored 43 using one or more of barbs or sutures or adhesives.
Electrochemical sensor 192B: The sensor 192 detects local chemical related changes. The sensor 192 may be anchored 43 using one or more of barbs or sutures 44 or adhesives.
Positional Sensor 192C: relative positions of two sensors 192 may indicate relative location and movement over time. The sensor 192 may be anchored to known landmarks to evaluate relative movement of structures to evaluate pelvic floor structure relative position. The sensor 192 may be anchored using one or more of barbed protrusions or sutures 44 or adhesives.
EMG Sensor 192D: Two tethers 53 may be anchored into the muscle, using, for example barbs 43, or sutures 44.
Nerve recorders: In some embodiments of a nerve stimulation system, one nerve stimulation device 100 is used to record nerve activity to serve as input to another nerve stimulation device.

Illustrative embodiments use one or more miniature, self-contained and implantable, battery powered stimulators 100 (also referred to as a neuromodulation device 100). In various embodiments, this stimulator may be the stimulator described in U.S. patent application Ser. Nos. 16/185,285, 16/414,169, 18/225,129, and/or 18/225,130, each of which are incorporated herein by reference.

The stimulator directly contacts the appropriate peripheral target nerve located in the pelvis to provide direct neuromodulation, which is more precise and requires lower power than volume conduction, to achieve a desired clinical effect. The inventors have successfully tested the neuromodulation device 100 in an acute and chronic sheep model for the treatment of SUI and for the treatment of OAB. Furthermore, the inventors have successfully tested the neuromodulation device 100 in a rabbit model for FI.

In some embodiments, one or more neuromodulation devices 100—including both primary neuromodulation devices 100A and satellite neuromodulation devices 100B—may be configured to receive control signals from an external computing device 304 operated by a clinician, such as a physician programmer or a clinic-based workstation. Additionally or alternatively, therapy parameters, schedules, and feedback may be adjusted or monitored via a patient-controlled mobile device 302, such as a smartphone or tablet, through a secure wireless interface. This external control allows for convenient real-time adjustment of stimulation settings, patient-reported outcomes tracking, and longitudinal therapy optimization. Furthermore, in various embodiments, the primary neuromodulation device 100A may be configured to receive power wirelessly from an external wireless power source 306, such as a wearable inductive coil or radiofrequency (RF) transmitter, enabling sustained or rechargeable energy delivery without the need for percutaneous connectors or frequent surgical intervention.

Illustrative embodiments enable the simultaneous treatment of multiple female pelvic health disorders. To that end, the system may include multiple neuromodulation devices to simultaneously treat SUI, OAB, POP and FI, etc. by neuromodulating specific target nerves that are key to each of the disorders being addressed. Each neuromodulation device 100 is capable of delivering a variety of stimulation patterns (e.g. afferent and efferent) and sensing the nerve response to stimulation. Satellite neuromodulation devices (shown in black in FIG. 3A-3B) may receive induction power and stimulation commands from a principal neuromodulation device 100 (shown in white in FIG. 3A-3B). To that end, the primary neuromodulation device 100 may include a wireless power transmitter (e.g., a wireless power transmitter coil) and the satellite neuromodulation devices may include a wireless power receiver circuitry 455 (e.g., a wireless power receiver coil). Thus, the satellite neuromodulation devices 100 may not include a battery (also referred to as a batteryless device), thereby providing a reduced size. However, in some embodiments, the satellite neuromodulate devices may include the battery. As an example, the battery may be a 3 mAh battery (e.g., EnerSys Quallion 3, or Resolution 3 mAh battery or a smaller solid state lithium battery or any other technology that provides adequate power).

The battery-powered principal neuromodulation device 100 may receive sensing data from the satellites. The sensing data, which may include electromyography (EMG) to sense muscle contractions, electroneurography (ENG) to sense nerve response and/or accelerometers to sense motion, among other things, may be used by the principal neuromodulation device 100 in the closed-loop feedback control of stimulation and the coordination of the therapies. With the addition of sensing and inter-implant communication technology to the platform, the treatment of multiple conditions may be coordinated. Since the larger principal neuromodulation device 100 may contain a significant amount of processing power, it can serve as a hub for optimal pelvic health.

In some embodiments, pelvic organ prolapse (POP) is treated using a distributed neuromodulation architecture that includes a principal neuromodulation device 100 and one or more satellite neuromodulation devices. This configuration enables coordinated therapy across multiple pelvic nerves and muscle groups.

As shown in FIG. 3B the principal neuromodulation device 100A (white) is implanted on a larger pelvic nerve (e.g., 1-6 mm in diameter) and includes a processor, power source, and one or more wireless communication coils. In some embodiments, the principal device 100A includes three orthogonally arranged coils or a combination of a conventional coil with orthogonal double-D coils to enable efficient omnidirectional power and signal propagation. The principal device 100A is configured to deliver induction power and stimulation commands to a plurality of satellite neuromodulation devices (black), each positioned on smaller, distal nerve branches ranging from approximately 100 microns to 1.5 mm in diameter.

Each satellite neuromodulation device 100B includes individualized nerve engagement features configured to interface with specific nerve targets. In various embodiments, the devices may include a stationary, deformable, or movable arm that defines a chamber configured to couple to the appropriate nerve size. The chamber may be proportional to the target nerve diameter, such that, for example, a treatment for POP includes neuromodulation devices having chambers configured to receive nerves of a variety of sizes. In various embodiments, the chamber is sized relative to the target nerve so that it provides less than 4 kPa of pressure to the nerve as it is stimulated by an electrode in the chamber. Additionally, or alternatively, the chamber may be configured so as to not reduce a cross-sectional area of the nerve more than 10%. Preferably, the cross-sectional area of the nerve is not reduced, but the cross-sectional shape may be changed. In a similar manner, if the device 100 has a channel through which the nerve travels to get to the chamber, the channel preferably is sized to reduce the cross-sectional nerve size no more than 25%-30% at any point as the nerve travels through the channel. impart less than 6.7 kPa on the nerve to prevent structural damage to the nerve. For example, the channel preferably has a cracking pressure of less than 6.7 kPa. In various embodiments, the device 100 100 is configured so that less than 10 kPa or less than about 1.5 psi of pressure is applied to the nerve. Of course, some embodiments may provide higher pressures than disclosed herein.

Although various embodiments describe a device 100 for coupling to, and treating, nerves having diameters of between about 0.2 mm and about 4 mm, it should be understood that such devices and methods are scalable to accommodate a variety of different sized nerves. For example, one skilled in the art may use the disclosure herein to configure the device 100 to couple with larger nerves having diameters of between about 5 mm and about 8 mm. These devices may be configured for long-term coupling with the various nerve sizes without damaging the nerve (e.g., by applying pressure to the nerve that is less than 4 kPa). Thus, in various embodiments, the devices are sized appropriately for a respective target nerve for atraumatic coupling. Atraumatic coupling provides less than a 4 kPa sustained pressure on the nerve when the nerve is in the chamber (e.g., in the chamber for many hours, days, etc.). Furthermore, atraumatic coupling provides less than 6.7 kPa when passing through the channel (e.g., pass through the channel for a short time period of less than 1 minute).

As used herein, the term “coupling to a nerve” or “engaging a nerve” is intended to encompass not only direct attachment or interfacing with an isolated peripheral nerve, but also interfacing with anatomical structures that include the nerve within a broader complex. These structures may include, but are not limited to, neurovascular bundles, and connective tissue sheaths that surround or encapsulate the nerve.

In many anatomical locations, particularly in the pelvic region and at distal branches of peripheral nerves, the nerve is co-located with one or more adjacent blood vessels, lymphatic vessels, and fibrous or connective tissue elements. These components together form what is commonly referred to as a neurovascular bundle or nerve plexus. Accordingly, when an implantable neuromodulation device 100 such as a nerve clip or cuff is “coupled” to a nerve, it may in practice couple with a target structure that includes the nerve along with surrounding vascular and connective components.

The present disclosure recognizes that in such embodiments, the electrode array, housing, or fixation mechanism may contact or envelop tissue beyond the nerve alone. Such interfacing is still considered “nerve coupling” for the purposes of this disclosure, provided that the structure being engaged contains a nerve that is subject to stimulation or sensing. In some instances, the nerve may be only one of several components within the engaged tissue structure, and the precise positioning of the stimulation or sensing elements may be based on anatomical access, surgical approach, or device sizing.

Furthermore, in some cases the nerve within the target structure may comprise multiple small fascicles, which may be spatially separated or partially insulated by surrounding connective tissue. The present system accommodates anatomical complexity by allowing for electrode configurations, sizing ranges, and conformable materials capable of delivering effective neuromodulation even in the presence of heterogeneous or distributed fascicular anatomy.

Thus, all references in this application to coupling the device 100 to “a nerve,” “a target nerve,” or “nerve interfacing” should be understood to include coupling to any anatomical structure that includes the target nerve, whether isolated or embedded within a broader multi-tissue bundle.

As described above, in various embodiments, implantable neurostimulation devices 100 (e.g., nerve clips, cuffs, or clamps) are designed with specific geometries and mechanical features to interface with nerves or nerve-containing structures of different sizes. The nerve interface may target an isolated nerve, or alternatively a target structure that includes the nerve along with associated tissue such as blood vessels, connective tissue, or other neurovascular components, as discussed above. Accordingly, the size and geometry of the neurostimulation device 100 (e.g., the channel and/or the chamber) are selected to accommodate the cross-sectional area of the entire target structure, not just the nerve itself.

To support a wide range of anatomical targets, the system may include a plurality of stimulation devices 100 with different sizing profiles. These may be provided individually or as part of a kit tailored for particular clinical procedures or therapeutic indications (e.g., stress urinary incontinence, fecal incontinence, or pelvic organ prolapse). A typical kit may include multiple neurostimulation devices 100 with distinct channel and chamber geometries to accommodate the known range of target structure sizes for that specific indication.

Each neurostimulation device 100 comprises a channel region into which the nerve or bundle is initially inserted and a chamber region in which the structure ultimately rests. In some embodiments, the device 100 includes one or more movable or deformable arms made of flexible materials (e.g., silicone or elastomer) that allow the channel width to temporarily expand during insertion and then contract to a more stable configuration for chronic implantation.

The chamber is generally designed to match the cross-sectional area of the largest nerve or neurovascular bundle in the expected size range. In preferred embodiments, the fit between the device 100 and the nerve or bundle is close, with no more than approximately ±10% variance from the ideal cross-sectional area, thereby minimizing slippage while avoiding excessive compression. In solid-state chambers (non-flexible) neurostimulation device configurations, this 10% range may represent the allowable compression window for safe long-term placement without inducing nerve damage.

The following table presents exemplary channel and chamber sizing values for devices targeting different nerve or neurovascular bundle size ranges in accordance with illustrative embodiments for neuromodulation devices 100 (e.g., particularly those including one or more movable or deformable arms):

TABLE 2
Neuromodulation Device with Moveable and/or Deformable Arm

| Target Nerve/ | Channel minimum cross- | | |
|---|---|---|---|
| Bundle Size | sectional dimension (in | | |
| Diameter | closed configuration) | | Chamber Max size (+/−10%) |
| 100-400 μm | 60 | μm | Cross-sectional area of a nerve having a |
| | | | diameter of 400 um (~0.1257 mm²) |
| 500 μm-1 mm | 300 | μm | Cross-sectional area of a nerve having a |
| | | | diameter of 1 mm (~0.7854 mm²) |
| 1 mm-3 mm | 600 | μm | Cross-sectional area of a nerve having a |
| | | | diameter of 3 mm (~7.0686 mm²) |
| 4 mm-7 mm | 2.4 | mm | Cross-sectional area of a nerve having a |
| | | | diameter of 7 mm (~38.48 mm²) |
| 7 mm-10 mm | 4.2 | mm | Cross-sectional area of a nerve having a |

diameter of 10 mm (~78.54 mm²)

Table 2 presents exemplary channel and chamber sizing values for neuromodulation devices configured to engage target nerves or neurovascular bundles of varying size ranges, in accordance with illustrative embodiments. For devices 100 incorporating a movable or deformable arm, two dimensions are described: (1) a minimum channel cross-sectional dimension, which refers to the narrowest distance between two opposing internal surfaces of the device 100, and (2) a maximum chamber cross-sectional area in the device's 100 open configuration, which corresponds to the maximum target anatomy the device is designed to accommodate.

For example, a chamber dimensioned to accommodate a 400-micron-diameter nerve or neurovascular bundle in its open state may have a cross-sectional area equivalent to that of a 400-micron circle, computed using the formula π·r² (yielding approximately 0.126 mm², where r=200 microns). This area serves as a reference to ensure that the chamber can receive anatomical structures of comparable size.

Importantly, the chamber itself need not have a circular shape. In practice, the internal cross-section of the chamber is often non-regular, such as elliptical, polygonal, or asymmetric, depending on the device 100 geometry. Similarly, the target nerve or neurovascular bundle—though roughly circular in native state—can elastically deform into a non-circular shape without incurring damage, so long as the compressive forces remain within atraumatic thresholds. Accordingly, a structure with a circular cross-sectional area may be safely accommodated in a chamber of equal area but different shape, preserving function and minimizing risk of ischemia, demyelination, or other compression-induced injury.

This design flexibility allows the use of a single chamber geometry to effectively and safely engage a range of anatomical targets with varying shapes and minor size deviations, particularly when combined with deformable or biased arm elements that dynamically conform to the target structure.

Table 3 presents exemplary channel and chamber sizing values for devices 100 targeting different nerve or neurovascular bundle size ranges in accordance with illustrative embodiments for neuromodulation devices 100 (e.g., particularly those having fixed channels and/or chambers):

TABLE 3
Neuromodulation Device with Stationary Channel and/or Chamber

| Target Nerve/ | | | | |
|---|---|---|---|---|
| Bundle Size | | Channel Width | | Chamber size (+/−10%) |
| 100 | | 60 | μm | Cross-sectional area of a nerve having a |
| | | | | diameter of 100 μm |
| 200 | μm | 120 | μm | Cross-sectional area of a nerve having a |
| | | | | diameter of 200 μm |
| 300 | μm | 180 | μm | Cross-sectional area of a nerve having a |
| | | | | diameter of 300 μm |
| 400 | μm | 240 | μm | Cross-sectional area of a nerve having a |
| | | | | diameter of 400 μm |
| 500 | μm | 300 | μm | Cross-sectional area of a nerve having a |
| | | | | diameter of 500 μm |
| 600 | μm | 360 | μm | Cross-sectional area of a nerve having a |
| | | | | diameter of 600 μm |
| 700 | μm | 420 | μm | Cross-sectional area of a nerve having a |
| | | | | diameter of 700 μm |
| 800 | μm | 480 | μm | Cross-sectional area of a nerve having a |
| | | | | diameter of 800 μm |
| 900 | μm | 540 | μm | Cross-sectional area of a nerve having a |
| | | | | diameter of 900 μm |
| 1 | mm | 600 | μm | Cross-sectional area of a nerve having a |
| | | | | diameter of 1 mm |
| 1.5 | mm | 900 | μm | Cross-sectional area of a nerve having a |
| | | | | diameter of 1.5 mm |
| 2 | mm | 1.2 | mm | Cross-sectional area of a nerve having a |
| | | | | diameter of 2 mm |
| 2.5 | mm | 1.5 | mm | Cross-sectional area of a nerve having a |
| | | | | diameter of 2.5 mm |
| 3 | mm | 1.8 | mm | Cross-sectional area of a nerve having a |
| | | | | diameter of 3 mm |
| 3.5 | mm | 2.1 | mm | Cross-sectional area of a nerve having a |
| | | | | diameter of 3.5 mm |
| 4 | mm | 2.4 | mm | Cross-sectional area of a nerve having a |
| | | | | diameter of 4 mm |
| 5 | mm | 3 | mm | Cross-sectional area of a nerve having a |
| | | | | diameter of 5 mm |
| 6 | mm | 3.6 | mm | Cross-sectional area of a nerve having a |
| | | | | diameter of 6 mm |
| 7 | mm | 4.2 | mm | Cross-sectional area of a nerve having a |
| | | | | diameter of 7 mm |
| 8 | mm | 4.8 | mm | Cross-sectional area of a nerve having a |
| | | | | diameter of 8 mm |
| 9 | mm | 5.4 | mm | Cross-sectional area of a nerve having a |
| | | | | diameter of 9 mm |
| 10 | mm | 6 | mm | Cross-sectional area of a nerve having a |
| | | | | diameter of 10 mm |

The values in Tables 2 and 3 are intended to serve as representative examples and may be adjusted based on anatomical data, specific clinical application, or desired coupling mechanics. In embodiments using flexible silicone-based designs, the channel and chamber dimensions allow for secure retention of differently sized structures via compliant deformation of the arms or walls of the device 100.

Thus, the above noted stratification of target nerve or neurovascular bundle sizes and corresponding device 100 dimensions presented herein is provided by way of example. In various embodiments, the neuromodulation devices 100 may be configured with finer or broader size bands, or alternative stratification schemes depending on clinical need, manufacturing preference, or surgical approach. The disclosed sizing are not intended to limit various embodiments.

In some cases, surgical or anatomical variability may result in the nerve or target structure being slightly outside the preferred range. The device 100 may still be used in such circumstances, provided that mechanical deformation remains within the safe compression limits and do not impair tissue perfusion or nerve function.

The satellites 100B receive power and control signals from the principal device 100A and may deliver stimulation in response to closed-loop control signals coordinated centrally. Sensing modalities, either integrated into the satellites or implemented as stand-alone implanted sensors, provide physiological feedback to the principal device.

The system includes a range of sensors 192 including electromyography (EMG), electroneurography (ENG), accelerometers 77, electrochemical sensors, and strain sensors. In one embodiment, EMG sensors 192 include needle-like probes that embed into a muscle or clip-like structures that attach to muscle surfaces with an electrode on the underside. Accelerometer sensors 77 may be configured similarly to EMG sensors 192 but lack the surface electrode. Electrochemical sensors 192 may include multiple fine electrodes for detecting biochemical signals, while strain sensors 192 include two anchoring points and a stretchable sensing element that detects deformation or relative positional changes of tissue structures. These sensors 192 transmit data to the principal neuromodulation device, enabling adaptive closed-loop coordination of therapy.

The principal neuromodulation device 100A receives the physiological data and adjusts therapy delivery dynamically, including modulation of pulse timing, amplitude, and duty cycle across the satellite network. This configuration enables simultaneous, individualized stimulation of multiple nerves to restore muscular support and prevent or repair pelvic organ prolapse. The architecture may further be extended to coordinate treatment of coexisting conditions such as stress urinary incontinence (SUI), overactive bladder (OAB), and fecal incontinence (FI), using a unified feedback-based control framework.

Based on preliminary efficacy data obtained in animal models for SUI, OAB, and FI, this multi-node closed-loop system is expected to provide superior therapeutic coordination. The processing capabilities of the principal device 100A allow for real-time sensor 192 interfacing, data analysis, and precise control of distributed therapy, thereby serving as a central hub for optimal neuromodulation-based pelvic health management.

For the sake of clarity in the description and figures, various embodiments may refer to a principal neuromodulation device as device 100A and one or more satellite neuromodulation devices as device 100B, in order to distinguish between their respective roles and features within a distributed architecture. However, unless otherwise specified, the general reference device 100 is used throughout the application to refer generically to any neuromodulation device, regardless of its specific configuration, capabilities, or role within a system. Thus, the description of device 100 is intended to encompass principal, satellite, standalone, or otherwise coordinated neuromodulation devices as appropriate in the given context. The use of specific lettered suffixes (e.g., 100A, 100B) is for illustrative convenience only and should not be interpreted as limiting the scope of the disclosure or claims.

In some embodiments, population and patient-specific data are collected over time to create a database to train an AI-controlled system.

Various embodiments may include one or more controllers 400. In one embodiment, the controller 400 is implanted within a patient 102 (e.g., within the principal neuromodulation device). In other embodiments, the controller 400 is within an external device 100 (e.g. a wearable controller 400, watch, or cell phone).

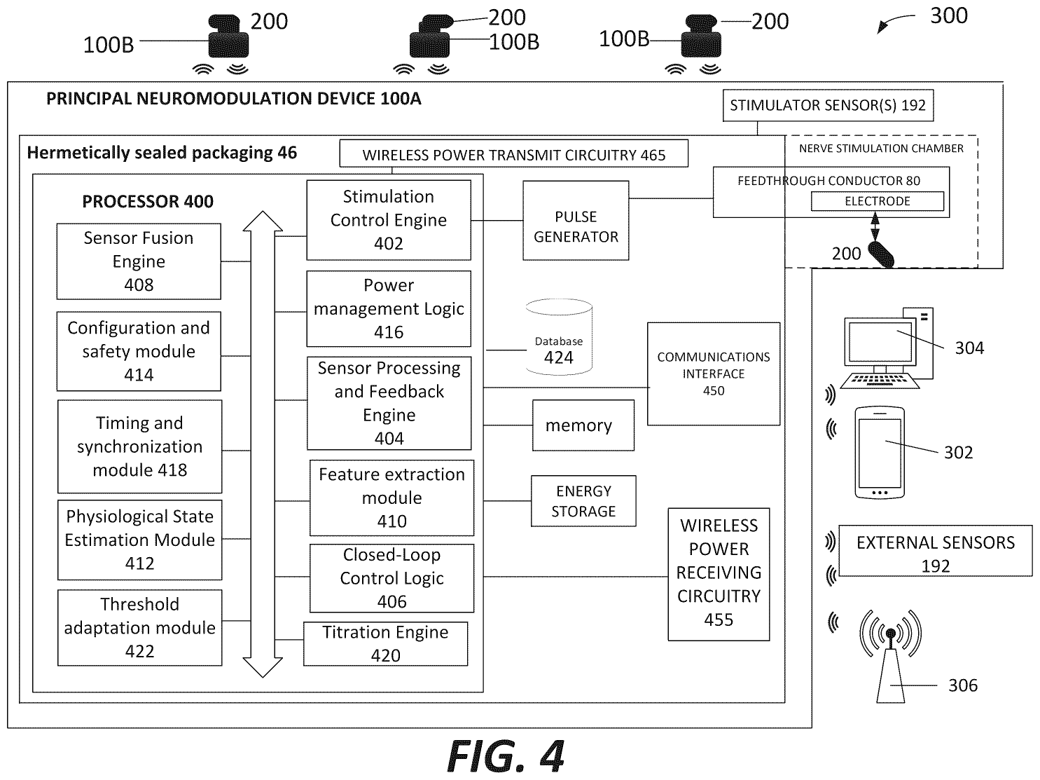

FIG. 4 schematically shows details of the neuromodulation device 100 controller 400 in accordance with illustrative embodiments of the invention. The controller 400 may be physically housed within the housing of the neuromodulation device 100, for example, in the primary neuromodulation device 100A. Each of the components in FIG. 4 operatively connected by any conventional interconnect mechanism. FIG. 4 simply shows a bus communicating each the components. Those skilled in the art should understand that this generalized representation can be modified to include other conventional direct or indirect connections. Accordingly, discussion of a bus is not intended to limit various embodiments.

Indeed, it should be noted that FIG. 4 only schematically shows each of these components. Those skilled in the art should understand that each of these components can be implemented in a variety of conventional manners, such as by using hardware, software, or a combination of hardware and software, across one or more other functional components. For example, the stimulator (discussed in detail below) may be implemented using a plurality of microprocessors executing firmware. As another example, the feedback analyzer may be implemented using one or more application specific integrated circuits (i.e., “ASICs”) and related software, or a combination of ASICs, discrete electronic components (e.g., integrated circuits), and microprocessors. Accordingly, the representation of the feedback analyzer and other components in a single box of FIG. 4 is for simplicity purposes only. In fact, in some embodiments, the feedback analyzer of FIG. 4 is distributed across a plurality of different components—not necessarily within the same housing or chassis.

It should be reiterated that the representation of FIG. 4 is a significantly simplified representation of an actual controller 400. Those skilled in the art should understand that such a device 100 has other physical and/or functional components, such as central processing units, other packet processing modules, and short-term memory. Accordingly, this discussion is not intended to suggest that FIG. 4 represents all of the elements of the controller 400.

As shown in FIG. 4, the neuromodulation device 100 can include a sensor 192 interface that interfaces with the sensors, a data storage, a network interface, a user interface, at least one battery (in some embodiments, the battery may be omitted—for example in the satellite, a wireless power transmitter 465 (e.g., in a primary device 100A) and/or a wireless power receiver 455 (e.g., in a satellite device 100B), wireless communication, a stimulator, and at least one processor.

In various embodiments, at least one principal neuromodulation device and one or more satellite neuromodulation devices, each designed to perform complementary roles in a multi-site, closed-loop stimulation framework. These devices may share a set of common internal components but differ in complexity, processing capacity, and stimulation control responsibilities depending on their respective roles in the system.

In various embodiments, both the principal 100A and satellite devices 100B have the following internal components:
Pulse Generator: A core functional unit that generates electrical stimulation waveforms with specified parameters such as amplitude, frequency, pulse width, and duration. The pulse generator converts digital or analog instructions into current or voltage pulses delivered to the nerve via electrodes. In various embodiments, each satellite neuromodulation device 100B comprises a local pulse generator configured to receive stimulation commands from the principal neuromodulation device and generate corresponding electrical stimulation waveforms for delivery to a target peripheral nerve.
Stimulation Circuitry: Includes the necessary analog drivers, charge-balancing circuits, isolation switches, and safety monitors required to deliver safe and effective stimulation. The circuitry ensures precise timing and amplitude control, minimizes leakage current, and maintains compliance with bioelectrical safety standards.
Electrode Interface: A conductive interface coupled to the nerve tissue, such as a bipolar or monopolar electrode array integrated into a flexible cuff, clip, or injectable lead. This interface transmits stimulation pulses from the circuitry to the target nerve and, in some cases, receives bioelectrical signals when used for sensing.
Communication Interface: A wireless module comprising a radio frequency (RF) transceiver, antenna or coil, and communication protocol controller 400 (e.g., BLE, NFC, custom telemetry). This interface supports two-way communication between the principal and satellite devices, and optionally with external systems such as clinician consoles or mobile apps.
Power Receiving Circuitry: An inductive receiver coil or capacitive coupling interface that allows the device to harvest energy from an external or implanted source. Includes rectifiers, voltage regulators, and protection circuitry to ensure stable power delivery under varying load conditions.
Energy Storage Module (optional): May include a rechargeable battery, supercapacitor, or charge reservoir to buffer harvested energy for delivery during active stimulation periods, ensuring smooth operation even under intermittent or variable wireless power availability.
Biocompatible Packaging: A hermetically sealed housing made of ceramic, titanium, silicone, or polymer encapsulant that protects internal electronics from body fluids and mechanical stress while ensuring safe chronic implantation.

In addition to the components above, the principal neuromodulation device 100A includes several advanced modules that support system-wide control, coordination, and decision-making:
Processor/controller 400: A microcontroller, application-specific integrated circuit (ASIC), or system-on-chip (SoC) that executes control algorithms, closed-loop logic, and stimulation coordination routines. It processes incoming biosignals, computes adaptive parameter updates, and issues commands to the satellite devices.
Non-Volatile Memory: Stores firmware, stimulation presets, biosignal history, calibration data, session logs, and patient-specific therapy settings. May include flash memory or magnetoresistive RAM (MRAM) for long-term retention.
Wireless Power Transmission Module: Comprising one or more transmission coils, such as three orthogonally arranged coils or a double-D coil configuration, this module enables efficient omnidirectional inductive power delivery to satellite devices implanted at different orientations or anatomical positions.
Sensor Aggregation Hub: Interfaces with one or more implanted or external sensors, including EMG, ENG, strain, pressure, accelerometers, and electrochemical sensors. Collects and digitizes biosignals for processing by the controller 400.
Telemetry Link to External Systems: Enables secure communication with an external controller, which may be a handheld programmer, clinician tablet, or patient mobile phone. Supports over-the-air firmware updates, therapy adjustments, and data export.
Time Synchronization Logic: Maintains synchronized clocks and stimulation timing coordination across multiple satellite devices, allowing for temporally controlled stimulation routines (e.g., alternating pulses, burst sequences, or phased contraction strategies).

The satellite neuromodulation devices 100B are miniaturized, peripheral implants responsible for localized stimulation and, in some embodiments, sensing. In addition to shared components, each satellite 100B may include:
Miniaturized Local controller 400: A reduced-size controller 400 (e.g., ASIC or minimal microcontroller) capable of interpreting incoming commands from the principal device and executing the corresponding stimulation waveforms locally.
Minimal Memory (optional): May include volatile or non-volatile memory sufficient to store current session parameters, identifiers, or temporary biosignal flags.
Local Sensing Module): Some satellites may include an embedded EMG electrode, ENG pickup, impedance sensor, or tissue strain sensor. These signals may be transmitted upstream to the principal device or used to adjust stimulation locally.
Current Steering or Multi-Contact Control Circuitry (optional): If equipped with multiple electrode contacts, the satellite may include switching logic or analog multiplexers to direct stimulation current to specific fascicles or contact zones.
Voltage and Current Regulators: Ensure consistent stimulation amplitude regardless of supply fluctuations due to variable wireless power coupling. May also enforce safety thresholds such as maximum charge per phase or duty cycle limits.
Internal Safety Watchdog: A circuit that disables stimulation if abnormal conditions are detected (e.g., temperature rise, excess current, or device malfunction).

Collectively, this distributed architecture enables fine-grained stimulation control across multiple pelvic nerves, real-time adaptation to biosignals, and modular expansion for additional conditions. The system's ability to balance control complexity in the principal device with localized pulse execution in the satellites allows for an optimized tradeoff between functionality, power efficiency, and miniaturization of the distal implants.

In various embodiments, one or more of the functional modules described herein—such as the Signal Acquisition Module, Feature Extraction Module, Physiological State Estimation Module, Titration Engine, Closed-Loop Controller, or Communication Interface—may be implemented wholly or partially on external systems, including a clinician-facing external computing device 304 or a patient mobile device 302. These external devices may serve as control hubs for therapy planning, remote monitoring, or longitudinal data analysis, and may operate in tandem with implanted components to facilitate adaptive and personalized therapy. Additionally, or alternatively, one or more of the modules may be distributed to or duplicated within satellite neuromodulation devices 100B. For example, a satellite device may locally process sensor data, execute simplified closed-loop routines, or implement fallback control algorithms when communication with the primary device 100A is temporarily lost. This flexible distribution of control logic allows the system to maintain functionality across a range of deployment scenarios, from fully centralized to distributed architectures.

In various embodiments, the satellite neuromodulation device 100B may lack certain higher-level functional modules present in the primary neuromodulation device 100A. For example, satellite devices 100B may omit the Physiological State Estimation Module, Titration Engine, and Database, instead relying on the primary device 100A or an external system (e.g., computing device 304 or mobile device 302) to perform those advanced analytics and long-term therapy adaptations. Satellite devices may also forgo a Comprehensive Communication Interface, limiting their interactions to local, device-to-device communication with the primary device 100A. Additionally, satellite devices 100B may not include full Closed-Loop Control Logic, instead executing basic instructions from the primary device without independently analyzing sensor data or determining stimulation adjustments. In some embodiments, satellite devices may also omit a dedicated power source such as a battery and instead operate using wireless power received from the primary neuromodulation device 100A. By simplifying onboard functionality, satellite devices 100B can be made smaller, more power-efficient, and better suited for distal nerve targets, while still participating in a coordinated and adaptive therapy regimen governed by the primary controller (e.g., in the primary device 100A or external computing device 304). This enables satellite devices 100B to be implanted in anatomical locations where larger or more complex neuromodulation systems may not be feasible or safe. This size advantage allows for precise targeting of distal or otherwise difficult-to-access nerves, particularly within the pelvic region or other confined spaces.

The data storage can include one or more of non-transitory computer readable media, such as flash memory, solid state memory, magnetic memory, optical memory, cache memory, combinations thereof, and others. The data storage can be configured to store executable instructions and data used for operation of the controller 400. In certain implementations, the data storage can include executable instructions that, when executed, are configured to cause the processor to perform one or more functions.

In some examples, the wireless communication interface can facilitate the communication of information between the controller 400 and one or more other devices or entities over a communications network. For example, where the controller 400 is included in the primary neuromodulation device 100 100, the network interface can be configured to communicate with a remote neuromodulation device 100 such as a satellite neuromodulation device 100 or other computing device. The wireless communication interface can include communications circuitry for transmitting data in accordance with a Bluetooth® wireless standard for exchanging such data over short distances to an intermediary device(s) (e.g., other neuromodulation devices). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface can include one or more physical interface devices such as input devices, output devices, and combination input/output devices and a software stack configured to drive operation of the devices. These user interface elements may render visual, audio, and/or tactile content. Thus, the user interface 219 may receive input or provide output, thereby enabling a user to interact with the controller 400 (e.g., via a smartphone application).

The controller 400 can also include at least one battery configured to provide power to one or more components integrated in the controller 400. The battery can include a rechargeable multi-cell battery pack. In one example implementation, the battery can include lithium ion cells that provide electrical power to the other device 100 components within the controller 400. The device 100 may include a wireless power transmitted and/or a wireless power receiver for charging the battery of the device, and/or wirelessly charging the battery of an associated device.

The sensor 192 interface may communicate with one or more sensors 192 (e.g., EMG, ENG, NCV, pressure sensors, etc.) configured to monitor, quantify, and/or detect one or more parameters of the patient 102 condition and/or neuromuscular response. As shown, the sensors 192 may be coupled to the controller 400 via a wired or wireless connection.

The sensor 192 interface can be coupled to any one or combination of sensors 192 to receive other patient 102 data indicative of patient 102 neuromuscular parameters. After data from the sensors 192 has been received by the sensor 192 interface, the data can be directed by the processor to an appropriate component within the controller 400. For example, the sensor 192 interface can transmit the data to the processor which, in turn, relays the data to a feedback analyzer. The data can also be stored on the data storage.

In various embodiments, the principal neuromodulation device 100A includes a processor or controller 400 configured to manage and coordinate all aspects of therapy delivery, sensor integration, communication, and closed-loop feedback. The controller 400 may be implemented as a microcontroller (MCU), a system-on-chip (SoC), a digital signal processor (DSP), or an application-specific integrated circuit (ASIC), depending on the complexity of the implementation and the desired balance of power efficiency, processing speed, and integration density.

The controller 400 may include or interface with a variety of functional modules and logic engines to support adaptive, closed-loop therapy in real-time and over extended periods. These functional components may include, but are not limited to the following components.

A stimulation control engine 402 manages the generation and timing of stimulation pulses delivered through the system. It determines:
When to initiate or terminate stimulation,
Which nerve or electrode contact to activate,
What waveform characteristics to apply, including pulse amplitude, pulse width, frequency, train duration, and inter-train intervals.
   The stimulation control engine 402 may control the delivery of phased or staggered delivery patterns for multiple target sites. In some embodiments, this module supports complex routines such as alternating bursts, phased bilateral stimulation, or dynamically varied pulse trains. The stimulation control engine 402 interfaces with local or remote pulse generators located in satellite neuromodulation devices and may adapt its outputs based on sensor data, user input, or preprogrammed schedules.

A Sensor Processing and Feedback Engine 404 receives input from one or more implanted or external sensors, including but not limited to:
Electromyographic (EMG) sensors, to assess muscle activation strength and timing
Electroneurographic (ENG) sensors, to detect neural conduction or latency
Strain sensors, to measure tissue displacement or structural engagement
Bladder or rectal pressure sensors, for bladder or rectal pressure
Electrochemical sensors, for metabolic or ionic changes
   The sensor processing and feedback engine 404 digitizes and filters the raw data (e.g., bandpass filtering of EMG in the 50-500 Hz range), then extracts key features such as peak amplitude, onset latency, contraction duration, or signal variance. These features are passed to the Closed-Loop Control Logic 406 for further interpretation.

A Closed-Loop Control Logic 406 performs real-time or session-based adjustments to stimulation parameters based on sensor feedback. Closed-loop actions may include:
Automatically reducing stimulation amplitude if muscle fatigue is detected (e.g., >20% EMG drop),
Increasing frequency or train count in response to improved responsiveness or strength,
Shifting stimulation focus from one nerve to another if asymmetrical recovery is detected.
   The closed-loop logic may operate on a per-train, per-session, or longitudinal basis, and can be configured to maintain therapeutic effectiveness while minimizing the risk of overstimulation or patient discomfort. The Closed-Loop Control Logic 406 may escalate therapy routines as muscle strength improves. Furthermore, it may compensate for asymmetrical recovery across multiple nerves.

Some embodiments include a multi-modal sensor fusion engine 408 to achieve a more comprehensive understanding of physiological states. For example:
EMG spikes may be correlated with strain displacement to confirm muscle engagement,
Bladder pressure may be analyzed alongside ENG activity to assess sphincteric coordination.
   The sensor fusion engine 408 may apply weighting schemes, temporal alignment algorithms, or heuristic models to interpret sensor data across different modalities and guide more precise therapy adjustments.

Communication Interface 450 and handles all data exchange between the principal device, satellite devices, and external systems. This includes:
Transmission of stimulation commands and parameter updates to satellite neuromodulation devices 100B,
Reception of sensor data or acknowledgments from satellites,
Secure wireless communication with external controller 400s, such as a clinician programmer, mobile phone, or cloud-connected hub.
Implanted Communications: Sends commands to and receives acknowledgments or sensor data from satellite devices using RF or near-field telemetry
External communication: Exchanges session data, logs, and therapy parameters with external devices (e.g., mobile app 302, clinician computing device 304, base station)
   Supported communication protocols may include Bluetooth Low Energy (BLE), near-field communication (NFC), or custom RF telemetry. The Communications Interface may also Encrypt and authenticates data as needed

The controller 400 may include a timing and synchronization module 418 to coordinate pulse delivery across multiple sites. This may involve:
Maintaining a master clock or timestamp system,
Scheduling stimulation trains with defined interleaving or phasing,
Ensuring Synchronized pulses (e.g., bilateral contraction) or alternating activation between different pelvic muscle groups.

A Database 424 may store longitudinal data for the controller 400 to analyze. The data may represent:
Session-level stimulation parameters,
Biosignal trends over time,
Longitudinal adaptation (e.g., progressive increase of stimulation intensity as performance improves)
Personalized baselining
Session-to-session optimization routines
Data export for clinician review
Patient-reported outcomes or subjective feedback.
   This information may be used to support long-term adaptation of therapy regimens, such as gradually increasing intensity as muscle strength improves or modifying the stimulation strategy in response to slow or asymmetrical recovery.

In various embodiments, the neuromodulation system includes a Titration Engine 420 a Threshold Adaptation Module 422, which work in conjunction to adjust stimulation parameters based on the evolving physiological state of the patient. The Threshold Adaptation Module 422 is configured to monitor and record stimulation-response characteristics over time, including motor thresholds, nerve conduction latency, and amplitude of evoked responses. Using data collected during therapy sessions or through periodic probe stimulations, the module tracks whether a given stimulation intensity continues to evoke the desired physiological response. If a nerve or muscle becomes more or less responsive (e.g., due to recovery, fatigue, or degradation), the module dynamically recalibrates threshold values accordingly. This adaptive threshold data is passed to the Titration Engine 420, which adjusts one or more stimulation parameters—such as amplitude, frequency, duty cycle, train duration, or pulse width—to optimize therapy delivery. For example, if a muscle demonstrates signs of strengthening (e.g., greater EMG amplitude at lower stimulation intensity), the Titration Engine 420 may increase stimulation intensity to further challenge the muscle and promote continued adaptation. Conversely, if fatigue or overstimulation is detected, the engine may reduce stimulation intensity or frequency to allow for recovery. This combined functionality ensures that therapy is not only safe and personalized, but also responsive to patient progress, thereby maximizing long-term therapeutic efficacy while minimizing side effects or energy waste.

Configuration and Safety Module 414 monitors system integrity and enforces safety constraints, such as:
Maximum allowable stimulation amplitude, frequency, or duty cycle,
Detection of abnormal tissue impedance, temperature, or signal noise,
Watchdog timers for device responsiveness and error recovery,
Failsafe shutdown protocols in the event of error or emergency conditions.
   The module may also provide fallback routines or “safe mode” stimulation settings that allow continued operation if sensor data becomes unreliable or unavailable.

Power Management Logic 416 Especially in fully implanted systems, power management is essential. This module may control:
Sleep and wake cycles based on stimulation demand or sensor activity,
Power budgeting across sensors, stimulators, and telemetry modules,
Monitoring of energy reserves in capacitors or rechargeable batteries,
Coordination of wireless energy harvesting windows with external or internal sources.

In some embodiments, the controller 400 includes a Feature extraction module 410 configured to compute signal metrics from biosignal inputs, including but not limited to peak amplitude, time-to-peak, waveform variance, signal envelope, and latency. These features are provided to a Threshold Adaptation Module 422 422, which tracks changes in response thresholds over time and adjusts therapy delivery in accordance with strengthening or fatigue. These components work in conjunction with the Closed-Loop Control Logic 406 and Sensor fusion engine 408 to provide precise, state-aware modulation of therapy parameters. In certain embodiments, elements of this control logic may be hierarchically distributed between the principal and satellite neuromodulation devices or integrated into an external computing platform.

In various embodiments, the neuromodulation system includes a Physiological State Estimation Module 412, which is a functional component of the controller or processor configured to evaluate the condition of a target nerve, neuromuscular junction, or muscle based on one or more biosignals. This module receives processed input from upstream signal acquisition and feature extraction modules—including electromyographic (EMG), electroneurographic (ENG), strain, or pressure sensors—and applies diagnostic logic to determine the current physiological state of the tissue. The module classifies the state into categories such as fatigued, weak, overstimulated, underactive, or strengthening. These classifications may be derived from absolute values, deviations from baseline, or signal trends observed across time. For example, the module may identify muscle fatigue based on a sustained decrease in EMG amplitude over repeated stimulation trains, or diagnose strengthening based on an increasing EMG response to consistent stimulation. Similarly, delayed ENG conduction latency may indicate nerve desensitization or under-recruitment. The outputs of this module inform the closed-loop control system and Titration Engine 420, enabling the system to personalize therapy delivery by adapting stimulation parameters in real time or over multiple sessions. In some embodiments, the module also integrates multiple signal modalities to refine diagnostic accuracy using sensor fusion logic.

These logic modules may be partially or wholly embedded within the principal device, and in some cases, simplified control routines may also reside within satellite devices to support limited autonomy. In some configurations, high-level coordination or parameter adjustment may be delegated to an external controller 400 (e.g., clinician programmer or patient mobile device), which operates in conjunction with the internal processor.

In some embodiments, a subset of this logic may also reside in satellite neuromodulation devices, enabling limited autonomous behavior (e.g., timing-based local stimulation) or local fallback behavior if communication with the principal controller 400 is temporarily lost. In other embodiments, high-level control may be distributed to an external device, such as a mobile phone, or clinician console, particularly for session planning, longitudinal trend analysis, or secure firmware updates.

In some implementations, the processor includes one or more processors (or one or more processor cores) that each are configured to perform a series of instructions that result in manipulated data and/or control the operation of the other components of the controller 400 and its various components. In some implementations, when executing a specific process (e.g., sending a signal to stimulate the patient), the processor can be configured to make specific logic-based determinations based on input data received, and be further configured to provide one or more outputs that can be used to control or otherwise inform subsequent processing to be carried out by the processor and/or other processors or circuitry with which the processor is communicatively coupled. Thus, the processor reacts to specific input stimulus in a specific way and generates a corresponding output based on that input stimulus. In some example cases, the processor can proceed through a sequence of logical transitions in which various internal register states and/or other bit cell states internal or external to the processor may be set to logic high or logic low.

As referred to herein, the processor can be configured to execute a function where software is stored in a data store coupled to the processor, the software being configured to cause the processor to proceed through a sequence of various logic decisions that result in the function being executed. The various components that are described herein as being executable by the processor can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

The stimulator can include, or be operably connected to, circuitry components that are configured to generate electrical signals through the electrode that may cause the stimulation to be transmitted to the patient 102 through the electrode. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the stimulator and under control of one or more processors to provide, for example, stimulation at a desired frequency, amplitude and/or pattern.

In some implementations, the processor includes one or more processors (or one or more processor cores) that each are configured to perform a series of instructions that result in manipulated data and/or control the operation of the other components of the controller 400 and the device 100. In some implementations, when executing a specific process (e.g., EMG detection, controlling the stimulator), the processor can be configured to make specific logic-based determinations based on input data received, and be further configured to provide one or more outputs that can be used to control or otherwise inform subsequent processing to be carried out by the processor and/or other processors or circuitry with which processor is communicatively coupled. Thus, the processor reacts to specific input stimulus in a specific way and generates a corresponding output based on that input stimulus. In some example cases, the processor can proceed through a sequence of logical transitions in which various internal register states and/or other bit cell states internal or external to the processor may be set to logic high or logic low.

As referred to herein, the processor can be configured to execute a function where software is stored in a data store coupled to the processor, the software being configured to cause the processor to proceed through a sequence of various logic decisions that result in the function being executed. The various components that are described herein as being executable by the processor can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

The most common control scheme is a PID (proportional, integral, differential) controller 400. The PID controller 400 may anticipate a single input and provide a single output. In embodiments having multiple satellite neuromodulation devices, the system may have multiple sensors 192 and therefore multiple inputs. These can be configured into a MISO PID controller 400—multiple inputs, single output. Illustrative embodiments may be configured to MISO PID controller 400 in multiple. As one example, each input may be in its own PID loop with a single output. As another example, the single outputs can have a weighted combination to determine the single output for the entire MISO PID control loop (as utilized with machine learning and AI techniques). It should be understood that within the treatment therapy, the inputs of the controller 400 may include the sensed data, and the output may include an adjustment in the course of therapy.

In illustrative embodiments, examples of outputs include stimulation current, stimulation voltage, stimulation frequency, stimulation pulse duration, latent time between stimulation pulses, number of stimulation pulses in a row and periodic clock time of stimulation. A key to getting such a control system to work optimally is to collect enough data to determine the weights to assign to each of the inputs in order to create the optimal outputs (i.e. train the model).

Although one type of control system was disclosed above, it should be understood that there are multiple control schemes that can be employed, including:
Linear Quadratic Regulator-minimizes an optimization function.
Adaptive Control
C Robust Control.
Fuzzy Logic Control

And various nonlinear control schemes. Many of the implementable nonlinear control schemes are just linear control schemes used in a small window of operation where the nonlinear can be approximated by the linear.

A number of feedback control schemes may use machine learning or AI. Examples of other control schemes of various embodiments may include:

Reinforcement Learning (RL): Reinforcement learning is a machine learning technique where an agent learns to make decisions by interacting with an environment to maximize a cumulative reward signal. In the context of control, RL algorithms learn optimal control policies by trial and error, adjusting their actions based on feedback from the environment. Stimulation parameters in an RL control system can be constrained individually or in combination (e.g. the product of amplitude*time shall not exceed X) to ensure that outputs remain safe as the model learns.

Deep Reinforcement Learning (DRL): Deep reinforcement learning extends RL by using deep neural networks to approximate complex control policies. DRL algorithms, such as Deep Q-Networks (DQN), Deep Deterministic Policy Gradient (DDPG), and Proximal Policy Optimization (PPO).

Neuroevolution: Neuroevolution is a technique that uses evolutionary algorithms, such as genetic algorithms or genetic programming, to evolve neural network controllers 400s for control tasks. By evolving the structure and parameters of neural networks over generations, neuroevolution can find effective control policies for complex systems.

Fuzzy Logic Control with Machine Learning: Fuzzy logic control (FLC) is a rule-based control method that uses linguistic variables and fuzzy rules to represent control policies. Machine learning techniques, such as genetic algorithms or neural networks, can be used to automatically optimize the fuzzy rule sets or tune the membership functions based on training data, improving the controller's 400 performance.

Model Predictive Control (MPC) with Machine Learning: Model predictive control (MPC) is a control technique that solves an optimization problem at each time step to generate control actions based on a dynamic model of the system. Machine learning techniques can be used to learn or approximate the system dynamics, making MPC more robust to model uncertainties and disturbances.

Meta-Learning for Control: Meta-learning algorithms learn from previous experience and adapt quickly to new tasks or environments. In the context of control, meta-learning can be used to train controllers that can generalize across different systems or adapt to changes in the system dynamics.

The principal neuromodulation device 100 and the satellite neuromodulation devices may communicate with wires or wirelessly. Additionally, the satellite neuromodulation devices may communicate with one another with wires or wirelessly. The communication protocols may include, among other things, microwave, RF, magnetic induction, ultrasound, Bluetooth (e.g., communicate over microwaves (2.5 Ghz), and/or communication through signal generation and impedance detection (e.g., it is possible to communicate through relative signal transmission and measurement of impedance signals).

The neuromodulation devices 100 described herein may include some or all of the features of the neuromodulation device 100 describes in in U.S. patent application Ser. Nos. 16/185,285, 16/414,169, 18/225, and/or 18/225,130. Each of these patent applications is incorporated herein by reference in its entirety. Furthermore, any of the features discussed herein may be integrated with any of the aforementioned neuromodulation devices 100. A brief description of the neuromodulation device 100 in accordance with illustrative embodiments is described in the appendix submitted herewith.

Over 90 million women in the US have pelvic floor disorders. Illustrative embodiments provide optimal treatment a much higher quality of life. A patient 102 utilizing similar embodiments has a better experience because the therapy may be: a) the first and final solution (which appeals to younger patients); b) a single procedure to treat any combination of pelvic floor disorders (OAB, SUI, Mixed Urinary Incontinence, POP, etc.); c) effective quickly; d) low to no pain; and e not contraindicated for women seeking to become pregnant. Clinicians may offer their patients the ability to treat multiple pelvic floor disorders in a single, easy-to-perform “cut down” surgical procedure that accesses peripheral nerves just under the dermis. Patient 102 progress may be monitored by an external patient 102 monitor that relays sensing and stimulation data back to the clinician.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

In some implementations, the processor 400 includes one or more processors (or one or more processor cores) that each are configured to perform a series of instructions that result in manipulated data and/or control the operation of the other components of the controller 400. In some implementations, when executing a specific process (e.g., nerve stimulation), the processor 400 can be configured to make specific logic-based determinations based on input data received, and be further configured to provide one or more outputs that can be used to control or otherwise inform subsequent processing to be carried out by the processor 400 and/or other processors or circuitry with which processor 400 is communicatively coupled. Thus, the processor 400 reacts to specific input stimulus in a specific way and generates a corresponding output based on that input stimulus. In some example cases, the processor 400 can proceed through a sequence of logical transitions in which various internal register states and/or other bit cell states internal or external to the processor 400 may be set to logic high or logic low. As referred to herein, the processor 400 can be configured to execute a function where software is stored in a data store coupled to the processor 400, the software being configured to cause the processor 400 to proceed through a sequence of various logic decisions that result in the function being executed. The various components that are described herein as being executable by the processor 400 can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

As used in this specification and the claims, the singular forms “a,” “an,” and “the” refer to plural referents unless the context clearly dictates otherwise. Thus, in various embodiments, any reference to the singular includes a plurality, and any reference to more than one component can include the singular. For example, reference to “the neuromodulation device” in the singular includes a plurality of neuromodulation devices, and reference to “the sensor” in the singular includes one or more sensors and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the subject matter contained herein has been described in detail for the purpose of illustration, it is to be understood that such detail is solely for that purpose and that the present disclosure is not limited to the disclosed embodiments, but, on the contrary, is intended to cover modifications and other arrangements that are within the spirit and scope of the appended claims. For example, it is to be understood that the present disclosure contemplates that, to the extent possible, one or more features of any embodiment can be combined with one or more features of any other embodiment.

What is claimed is:

1. A closed-loop neuromodulation system comprising:
(a) at least one implantable neurostimulator configured to deliver electrical stimulation to a target nerve innervating a muscle;
(b) at least one biosignal sensor configured to detect a physiological signal associated with the target muscle, the physiological signal comprising an electromyographic (EMG) or electroneurographic (ENG) signal;
(c) a controller operatively coupled to the neurostimulator and the sensor, the controller configured to:
(i) receive the physiological signal from the sensor;
(ii) determining whether the physiological signal satisfies a predefined condition indicative of weakness, strength, fatigue, muscle underactivity, or overstimulation; and
(iii) in response to determining that the condition is satisfied, automatically adjust one or more stimulation parameters selected from amplitude, frequency, pulse width, train duration, or train count.

2. The system of claim 1, wherein the sensor comprises an intramuscular electrode configured to record EMG activity from the pubococcygeus, puborectalis, or iliococcygeus muscle.

3. The system of claim 1, wherein the predefined condition comprises a decrease in EMG envelope amplitude of 20% or more during stimulation.

4. The system of claim 1, wherein the controller is further configured to reduce stimulation amplitude to 25-50% of a baseline threshold upon detection of the predefined condition.

5. The system of claim 1, wherein the stimulation is configured to treat pelvic organ prolapse (POP) by targeting nerves that innervate muscles selected from the group consisting of the pubococcygeus, puborectalis, iliococcygeus, and coccygeus.

6. The system of claim 1, wherein the controller is configured to titrate stimulation levels over time based on progressive changes in the diagnosed physiological state.

7. The system of claim 1, wherein the system initiates a probe stimulation to determine a motor response threshold and adjusts stimulation based on longitudinal threshold changes.

8. The system of claim 1, wherein the diagnosis comprises evaluating changes in motor response thresholds to determine if the muscle has strengthened or fatigued.

9. A neuromodulation system comprising:
(a) a primary stimulator having a controller comprising communication circuitry;
(b) at least one implantable satellite stimulator, each configured to be coupled to a different peripheral nerve;
(c) wherein the primary controller is configured to wirelessly transmit power and control signals to the satellite stimulators;
(d) and wherein the at least one satellite stimulator and the primary simulator are configured to deliver electrical stimulation to respective nerves in a coordinated or independent manner.

10. The system of claim 9, wherein each satellite stimulator comprises a nerve clip electrode configured to engage a nerve with a fascicle count between one and five.

11. The system of claim 9, wherein the primary controller is configured to receive biosignal data from at least one of the satellite stimulators and adjust stimulation parameters for the corresponding or another satellite stimulator based on the biosignal data.

12. The system of claim 9, wherein the satellite stimulators are configured to deliver stimulation asynchronously to produce differential activation of the pelvic floor muscles.

13. The system of claim 9, wherein one satellite stimulator comprises a sensing module configured to detect EMG signals, and another satellite stimulator comprises only a stimulation module.

14. The system of claim 9, wherein the primary controller is further configured to execute a therapy routine for pelvic organ prolapse by independently activating stimulators associated with the pubococcygeus and puborectalis muscles.

15. A neuromodulation system for treating pelvic organ prolapse (POP), comprising:
(a) a first implantable stimulator configured to deliver electrical stimulation to a first nerve innervating a first pelvic floor muscle selected from the group consisting of the pubococcygeus and puborectalis;
(b) a second implantable stimulator configured to deliver electrical stimulation to a second nerve innervating a second pelvic floor muscle distinct from the first;
(c) a controller operatively coupled to the first and second stimulators and configured to coordinate stimulation delivery based on a predefined therapy regimen for POP.

16. The system of claim 15, wherein the controller is configured to alternate stimulation between the first and second stimulators to prevent synchronous overactivation and to promote balanced muscle engagement.

17. The system of claim 15, wherein the controller is further configured to decrease stimulation amplitude, pulse train count, or treatment frequency upon detecting a greater than 20% drop in EMG signal amplitude.

18. The system of claim 15, wherein the first and second nerves comprise two or more distinct pelvic region nerves selected from the group of perineal branches of the pudendal nerve and branches of the levator ani nerve.

19. The system of claim 15, wherein the stimulator is configured to engage a distal portion of the target nerve comprising fewer than five fascicles.

20. The system of claim 15, wherein the stimulation is delivered to a segment located within the distal third of the anatomical length of the nerve.
